(19) United States
(12) Patent Application Publication
(10) Pub. No.: US 2023/0251262 A1
(43) Pub. Date: Aug. 10, 2023

(54) ENRICHMENT, DETECTION AND CHARACTERIZATION OF CIRCULATING TUMOR CELLS WITH SUSD2 AND ENPP1

(71) Applicant: Universitaesklinikum Hamburg-Eppendorf, Hamburg (DE)

(72) Inventors: Parinaz Mossahebi Mohammadi, Hamburg (DE); Kai Bartkowiak, Grosshansdorf (DE); Klaus Pantel, Hamburg (DE)

(21) Appl. No.: 17/667,701

(22) Filed: Feb. 9, 2022

(51) Int. Cl. G01N 33/574; G01N 33/543; G01N 33/58

(52) U.S. Cl. CPC G01N 33/57492; G01N 33/582; G01N 33/54326

(57) ABSTRACT

A method for the isolation, or isolation and detection, of circulating tumor cells (CTCs) from blood or lymph, or disseminated tumor cells (DTCs) from bone marrow. SUSD2 or ENPPl is used as a biomarker for the isolation of CTCs or DTCs. Isolation can, for example, be done immunomagnetically using anti-SUSD2 or anti-ENPPl antibodies coupled to magnetic particles.

### BACKGROUND OF THE INVENTION

### Field of the Invention

The invention relates to an improved method for the isolation, and optionally including detection, of circulating tumor cells (CTCs) from blood or lymph and/or disseminated tumor cells (DTCs) from bone marrow. Isolation of CTCs and/or DTCs allows for a subsequent genetic or other analysis, e.g. for developing therapies.

### Description of the Related Art

The metastatic cascade in breast cancer comprises the release of primary breast tumour cells into the blood (circulating tumour cells, CTC) followed by the settlement of such tumour cells at secondary organs (disseminated tumour cells, DTC) and their later metastatic outgrowth. However, there is still a substantial number of patients who relapse despite negative CTC findings at primary diagnosis in breast cancer.

As used herein, the term CTC relates to a tumor cell in the peripheral blood or lymph and DTC relates to a tumor cell in bone marrow. CTCs and DTCs are extremely difficult to detect. They are exceptionally rare and may be difficult to distinguish from healthy cells. Existing approaches for detecting CTCs and DTCs have limitations in sensitivity and/or specificity, leading to many healthy cells being mischaracterized as cancerous, and many cancer cells being missed in the analysis.

The current established and used biomarkers for detection and enrichment of tumor cells from the blood or bone marrow of patients are strongly limited to epithelial properties and downregulated in mesenchymal tumor cells. Detection is done using the “epithelial cell adhesion molecule” (EpCAM) or other epithelial markers like cytokeratin (CK). Among a pool of epithelial CTCs there are tumor cells that have undergone or are undergoing “epithelial-to-mesenchymal transition” (EMT) and lost much of their epithelial phenotype (to allow them to detach from the primary tumor actively). Such CTCs and DTCs are thought to be major drivers in metastasis formation and express epithelial markers only in low levels, making their isolation or detection with these markers difficult. EpCAM is a transmembrane protein with an extracellular domain and is suitable both for the detection and the isolation of CTCs and DTCs expressing large enough levels of epithelial markers. In contrast, cytokeratins are cytoplasmic proteins and are only suitable for the detection of CTCs and DTCs, but not for the isolation of the cells.

WO2013036620A1 entitled “Methods and Compositions for Cytometric Detection of Rare Target Cells in a Sample” discloses cytometric methods for the detection of rare target cells, such as circulating tumor cells (CTCs), in a biological sample such as blood. Aspects of the methods include contacting the sample with first and second binding members that specifically bind to a marker of the rare target cell, and cytometrically assaying the sample for the presence of cells comprising bound first and second binding members to detect the rare target cell in the sample. While this publication describes some general approaches for the detection of rare cells in a sample, it is limited to detection. Samples are analyzed for the presence of the cells. There is no mention of any further processing of the sample, including isolation. In addition, neither Sushi domain-containing protein 2 (SUSD2) or Ectonucleotide pyrophosphatase/phosphodiesterase family member 1 (ENPP1) are mentioned here, nor the fact that for isolation of the cells at least one antibody has to bind to the extracellular domain of the target protein. In principle, two antibodies can also bind to a protein that is exclusively present in the cytoplasm. There is no attempt in this patent to differentiate between cytoplasmic (sole detection) and transmembrane (isolation and detection) proteins.

WO 2018185336 A1 entitled “Optofluidic device and method for detecting circulating tumour cells” discloses a microfluidic device for detection and quantification of CTCs. Therefore, the sample is incubated with two labelled probes that target different CTC surface marker. Signals corresponding to the labelled probes are then detected in the microfluidic device. SUSD2 is mentioned as a possible mesenchymal lineage marker. However, it is not mentioned that SUSD2 could be used for isolation of CTCs.

It is an object of the invention to provide an improved method for isolation, and preferably isolation and detection, of CTCs and DTCs from blood, lymph or bone marrow. Isolation of CTCs and DTCs allows for a subsequent genetic or other analysis, e.g., for developing therapies. The biomarker proposed by the invention might function as potential immune target for personalized therapies. For example, highly personalized approaches, like the chimeric antigen receptor T-cell (CAR-T) technology, are an option to specifically attack cancer cells while reducing unwanted side effects. CARs usually contain specificity-conferring extracellular antibody single chain variable fragment (scFv), a CD3z-domain and intracellular costimulatory domains. The procedure begins with the isolation of T-cells from the blood of a patient, followed by a genetic modification to recognize the surface protein of interest and re-injection into the donor.

In the histogenesis of breast cancer, different pathways for the development of cancer phenotypes are possible [13]. Stem/progenitor cells (mesenchymal phenotype) differentiate via intermediary cells (epithelial/mesenchymal phenotype) to glandular cells (epithelial phenotype). During these steps, the cells may acquire gene aberrations leading to breast cancer phenotypes varying in their degree of epithelial differentiation and oncoprotein expression. Therefore, an approach is desirable that enables the detection/isolation of representative tumor cells of these phenotypes irrespective of their degree of epithelial differentiation.

It is also an object of the invention to provide a method particularly suited for the isolation and preferably isolation plus detection of cells with epithelial and/or mesenchymal attributes, cells with mesenchymal attributes meaning cells showing only low level of keratin (like cells of the cell line MDA-MB-231) or almost negative for keratin like the DTC cell line from the bone marrow of a breast cancer patient BC-M1 [1]. CTCs or DTCs with mesenchymal or epithelial/mesenchymal phenotype are thought to be major drivers in metastasis formation. As the current established and used biomarkers (EpCAM, cytokeratins) for detection and enrichment of tumor cells from the blood of patients are strongly limited to epithelial properties and downregulated in mesenchymal tumor cells, the present invention aims to close this “detection gap” for tumor cells with mesenchymal attributes by identifying new cell surface biomarker proteins for a broad spectrum of different CTC/DTC subpopulations. However, the invention is not limited to the enrichment, isolation and/or detection of CTCs or DTCs with mesenchymal attributes, but is also applicable to the enrichment, isolation and/or detection of CTCs or DTCs with epithelial attributes.

### SUMMARY OF THE INVENTION

An analysis of a novel CTC-derived breast cancer cell line (CTC-ITB-01, [2]) revealed elevated expression of Sushi-domain-containing protein 2 (SUSD2) and Ectonucleotide pyrophosphatase/phosphodiesterase family member 1 (ENPP1) compared with the breast cancer cell line MCF-7 (FIG. 1A). For both proteins a tumor-promoting impact was described in breast cancer [3-5]. Expression of these proteins on different breast cancer cell lines was proven by Western Blot and immunofluorescent analyses. While the expression of SUSD2 was observed by cell lines of different phenotype, ENPP1 was also detected in the strongly mesenchymal DTC cell line BC-M1 (FIG. 1B).

To date, SUSD2 and ENPP1 were not described in the context of Liquid Biopsy or CTCs. However, both proteins were detected in solid tumors and their role in different cancer entities was analyzed [3-7]. The invention allows enrichment and isolation, and optionally including detection, of viable SUSD2-positive and ENPP1-positive CTCs or DTCs that express the protein on their cell surface using, for example, a direct magnetic labeling approach of the MACS® technology. Different indirect magnetic labeling approaches using SUSD2 as target were described in literature to isolate SUSD2-positive mesenchymal stem cells in human palatine tonsil, bone marrow and endometrium [8-10]. Enrichment and isolation of ENPPl-positive cells by means of the MACS® technology were not described.

In order to use transmembrane proteins as specific CTC or DTC biomarkers, they should not be expressed by peripheral blood mononuclear cells (PBMCs). To verify the suitability of the transmembrane proteins SUSD2 and ENPP1 as CTC biomarkers, the expression of these proteins in PBMCs was analyzed. Western Blot analyses showed no signal for SUSD2 and ENPP1 in PBMCs, confirming their suitability as CTC biomarkers. The expression of ENPP1 and SUSD2 was analyzed on 13 breast cancer cell lines and four prostate cancer cell lines. While ENPP1 was expressed by breast cancer cell lines with mesenchymal characteristics, the expression of SUSD2 was mainly observed in breast cancer cell lines of different phenotype. A strikingly high expression of SUSD2 was detected in CTC-ITB-01 and its corresponding mouse xenograft cell line (CTC-ITB-01 MIND), suggesting that this protein could play an important role in the establishment of CTC cell lines. The expression of ENPP1 and SUSD2 was also observed in prostate cancer cell lines.

Experimental results show that peripheral blood mononuclear cells (PBMC) are virtually negative for SUSD2 and ENPP1 (FIG. 2), whereas a subpopulation of breast cancer cells is positive for these proteins (FIG. 1). Therefore, these proteins are suitable as novel CTC detection marker proteins, which may expand the number of detectable CTC.

The core of the invention is the enrichment, isolation, detection and/or characterization of CTCs with novel biomarkers. For the first time, expression of SUSD2 and ENPP1 on patient CTCs was proven and thus their suitability as biomarkers in liquid biopsy analyses for the isolation and optionally isolation plus detection of CTCs and DTCs. The invention can, however, also be used in the context of other cancers with a high potential to form metastases, e.g., prostate cancer, pancreatic cancer, head and neck cancer, lung cancer or malignant mesothelioma.

On the one hand, the invention aims to enrich and preferably isolate a specific subpopulation of viable CTCs that can be further analyzed, especially in regards of the metastatic potential of such subpopulations. On the other hand, it can be used to increase the number of enriched CTCs and to isolate CTCs by using, for example, a combination of MicroBeads targeting commonly used enrichment markers such as EpCAM and new markers like SUSD2 and ENPP1. Furthermore, in combination with commonly used detection markers, SUSD2 and ENPP1 could improve the detection rate of CTCs and promote treatment selection by enhancing CTC characterization.

Isolation can, for example, be done via magnetic-activated cell sorting (MACS®), a method for separation of various cell populations depending on their surface antigens (CD molecules), using anti-SUSD2 or anti-ENPP1 antibodies. The inventors developed a specific MACS-based system for isolating CTCs or DTCs expressing SUSD2. The present invention also allows for the isolation of CTCs or DTCs expressing ENPP1 by this system. Isolation is, however, not limited to this method, and alternatively isolation can be done, for example, by a specially adapted commercial system called “CELLSEARCH” (see, for example, Coumans F, Terstappen L., 2015, Detection and Characterization of Circulating Tumor Cells by the CellSearch Approach, Methods Mol Biol. 1347:263-78, doi: 10.1007/978-1-4939-2990-0_18; Mesquita B, et al. 2017, Molecular analysis of single circulating tumour cells following long-term storage of clinical samples, Mol Oncol. 11(12):1687-1697. doi: 10.1002/1878-0261.12113) or by other methods, for example by immune cell isolation methods using immobilized anti-SUSD2 or anti-ENPP1 antibodies (e.g., anti-SUSD2 or anti-ENPP1 antibody coated matrices) or other binding molecules (e.g., aptamers) specifically binding to SUSD2 or ENPP1. Methods using, for example, antibody-coated microstructures are also encompassed by the invention.

In the case of isolation plus detection the present invention requires the contacting of the cells with at least 2 antibodies, for example:
1. capture with an anti-SUSD2 or anti-ENPP1 antibody targeting the extracellular domain of SUSD2 or ENPP1, followed by detection of the captured cells by a labeled secondary antibody targeting the bound anti-SUSD2 or anti-ENPP1 antibody, or
2. capture of the cells with a first anti-SUSD2 or anti-ENPP1 antibody targeting the extracellular domain of SUSD2 or ENPP1, followed by the detection of the captured cells by a labeled secondary antibody targeting a second anti-SUSD2 or anti-ENPP1 antibody, bound to the intracellular domain of SUSD2 or ENPP1. Alternatively, the second anti-SUSD2 or anti-ENPP1 antibody can itself be fluorescently labeled.

An anti-keratin antibody, i.e., an antibody directed against cytokeratin, may additionally be used for detecting cytokeratin-positive cells, thus to distinguish between CTCs and DTCs having a mesenchymal phenotype (mCTC, mDTC), which would not test cytokeratin-positive, and epithelial (eCTCs) and hybrid epithelial/mesenchymal phenotype (emCTCs, emDTCs), which would test cytokeratin-positive.

By way of comparison, U.S. Pat. 8,524,493 entitled “Released Cytokeratins as Markers for Epithelial Cells” teaches a protocol for the EPISPOT assay with the detection of the cells by released keratins. In this case, the cells are cultivated on a synthetic membrane. In contrast, MACS is performed in liquid solution, which allows subsequent handling of the cells. Moreover, the present invention is particularly suitable for the enrichment, isolation and preferably additionaly detection of cells with different attributes, including cells with mesenchymal attributes, i.e. cells that show only low level of keratin (MDA-MB-231) or are almost negative for keratin (BC-M1).

### BRIEF DESCRIPTION OF THE DRAWINGS

Further details and features of the invention will be described on the basis of the following figures, showing:

FIG. 1A Western blot analysis of CTC biomarker candidates. Investigation of SUSD2 and ENPPlexpression in breast cancer cell lines BC-M1, MDA-MB-468, MCF-7, CTC-ITB-01, Cama-1, T47D and KPL-1. α-tubulin was used as loading control (n=3),

FIG. 1B Determination of epithelial and mesenchymal characteristics of CTC-ITB-01 and the reference cell line candidates by Western blot analysis. The expression of the epithelial markers E-cadherin, EpCAM, K8, K18 and K19, the mesenchymal markers N-cadherin and vimentin, and the stem-cell marker CD44 was analyzed in CTC-ITB-01 and the reference cell line candidates. BC-M1 and MDA-MB-468 were applied as positive controls and α-tubulin was used as loading control. Twenty micrograms (40 µg for EpCAM) of total cell extract of each cell line was applied (n=3). K8, keratin 8; K18, keratin 18; K19, keratin 19,

FIG. 2 Western blot analysis of SUSD2 and ENPP1 in PBMCs: Expression of SUSD2 and ENPP1 in PBMCs was analyzed. Therefore, PBMCs lysates from five different healthy donors were prepared and subjected to SDS PAGE. BC-Ml, MDA-MB-231 B02 and MDA-MB-468 were used as positive controls. Forty micrograms of protein were applied to each lane. α-tubulin was used as loading control,

FIG. 3 Detection of ENPP1 on CTCs of patients with breast cancer: Cells were stained with anti-ENPP1 antibody on a glass slide. Furthermore, pan-keratin was used as tumor cell marker, CD45 as leukocyte marker and nuclei were visualized using DAPI. A: ENPP1-positive CTC of patient 1. B: ENPPl-negative CTC of patient 1. C: ENPP1 - positive CTC of patient 2. D: ENPPl-negative CTC of patient 2. Images were taken at 400 x magnification,

FIG. 4 Detection of SUSD2 expression on CTCs: Representative pictures of CTCs enriched from blood of patients with metastatic breast cancer using the CellSearch ® system. CTCs were identified with the CellSearch® CTC Kit as keratin+ /DAPI+ /CD45- cells. SUSD2 expression was investigated with the anti-SUSD2 Vio Bright-FITC antibody. A: SUSD2-negative CTC. B and C: CTCs with low SUSD2 expression. D and E: CTCs with moderate SUSD2 expression. F: CTC with strong SUSD2 expression,

FIG. 5 Domain structure of SUSD2 a) Full-length membrane-bound SUSD2. b) Predicted final version of membrane-bound SUSD2, cleaved within the extracellular domain and releasing a fragment, which binds via disulfide bonds to the remaining membrane-bound fragment,

FIG. 6 Domain structure of ENPP1 a) Full-length membrane-bound ENPP1. b) Soluble form of ENPP1, cleaved intracellularly,

FIG. 7 Simplified representation of capture and detection of SUSD2 or ENPP1 positive CTC by an immunomagnetic cell separation method, using two different approaches. SUSD2 is shown here as an example. In both approaches an anti-SUSD2 or ENPP1 antibody (“capturing” antibody), which is directed to the extracellular domain of SUSD2 or ENPP1, was coupled to magnetic nanoparticles. In the first approach (FIG. 7a) a labeled secondary antibody directed against the anti-SUSD2 or ENPP1 antibody or against the complex composed of the anti-SUSD2 or ENPP1 antibody, the magnetic nanoparticle and the components (e.g. biotin and streptavidin) coupling the magnetic nanoparticle and the anti-SUSD2 or ENPP1 antibody, is used to detect SUSD2⁺ or ENPP1⁺ CTCs, in the second approach (FIG. 7b) a second primary anti-SUSD2 or anti-ENPP1 antibody, directed against the intracellular domain of SUSD2 or ENPP1, and a secondary antibody directed against the second primary anti-SUSD2 or anti-ENPP1 antibody are used to detect SUSD2⁺ or ENPP1⁺ CTCs,

FIG. 8 Schematic illustration of an example of a cell surface molecule-dependent MACS isolation procedure. CTCs carrying the cell surface molecule (e.g., a transmembrane protein) are captured by an antibody directed against the cell surface molecule. The antibody is coupled to a magnetic nanoparticle, and the complex of antibody bound to the CTC and the magnetic nanoparticle can be retained in a column by a magnet (FIG. 8a) and subsequently released from the column by removing the magnet (FIG. 8b),

FIG. 9 Microscopic evaluation by immunofluorescent double staining of the MACS® system using SUSD2 MicroBeads and breast cancer cell line cells spiked into blood of healthy donors as model system. Cytospins were obtained after conducting MACS® with SUSD2 MicroBeads. Slides were stained with anti-mouse lgG to detect the MicroBeads bound tumor cells. Furthermore, pan-keratin was applied as tumor cell marker, CD45 as leukocyte marker and nuclei were visualized with DAPI. A: CTC-ITB-01 cell present in the elution fraction. B: CTC-ITB-01 cell, which was found in the combined flow - through and wash fraction. C: T47D cell in the elution fraction. D: T47D cell in the combined flow-through and wash fraction. White scale bars represent 20 µm. Images were taken at 400 x magnification,

FIG. 10 Microscopic analysis by immunofluorescent double staining of breast cancer patient CTCs obtained by MACS® using SUSD2 MicroBeads (elution fraction). The cytospin of the elution fraction was stained with anti-mouse IgG to detect CTCs bound by SUSD2 MicroBeads. Furthermore, pan-keratin was applied as tumor cell marker, CD45 as leukocyte marker and nuclei were visualized with DAPI. A: CTC cluster with SUSD2-positive CTCs. B: CTCs with positive and negative mouse-IgG staining. C: Zoom of CTCs with positive mouse IgG staining denoted by arrow C in image B. D: Zoom of CTCs with negative mouse IgG staining denoted by arrow D in image B. Images of the CTC cluster were taken at 400 x magnification and images in B at 200 x magnification. White scale bars represent 10 µm and blue scale bars correspond to 30 µm,

FIG. 11 CLUSTAL multiple sequence alignment of the amino acid sequences from human SUSD2 (SUSD2_HUMAN; UniProtKB Q9UGT4 (sequence version (SV) 1)) with the bioinformatics predicted SUSD2 proteins by sequence homology from canis familiaris (dog; UniProtKB E2RSH4 (SV3)), cavia porcellus (guinea pig; UniProtKB H0VMC5 (SV1)) and equus caballus (horse; UniProtKB F6QE14 (SV2)). The identity of the amino acid sequences compared with human SUSD2 are 76.59% (pig), 76.77 (dog), 78.56 (horse),

FIG. 12 CLUSTAL multiple sequence alignment of the amino acid sequences from human ENPP1 (ENPP1_HUMAN; UniProtKB P22413 (SV2)) with the bioinformatics predicted ENPP1 proteins by sequence homology from canis familiaris (dog; UniProtKB F1PJP0 (SV2)), cavia porcellus (guinea pig; UniProtKB H0VSW3 (SV2)) and equus caballus (horse; UniProtKB F7ALR7 (SV2)). The identity of the amino acid sequences compared with human ENPP1 are 79.22% (pig), 86.57 (dog), 86.49 (horse),

FIG. 13 MS survey scan of SUSD2 peptide CGALDGPCSCHPTCSGLGTCCLDFR detected in a MCF-7/CTC-ITB-01 SILAC sample. MS survey scans show the peak intensities on the vertical axes and the horizontal axes display the m/z. A: MS survey scan at a retention time of 35.14 minutes. Some peaks are only annotated for orientation. B: Enlarged section of the MS survey scan in A at a mass range of m/z = 950 - 960, showing the SUSD2 peptide with light masses from CTC-ITB-01. Monoisotopic peak of the light peptide ion is shown at m/z = 953.38 [M + 3 H]3+, and

FIG. 14: MS survey scan of ENPP1 peptide AEYLHTWGGLLPVISK detected in a MCF-7/CTC-ITB-01 SILAC sample MS survey scans show the peak intensities on the vertical axes and the horizontal axes display the m/z. A: MS survey scan at a retention time of 44.87 minutes. Some peaks are only annotated for orientation. B: Enlarged section of the MS survey scan in A at a mass range of m/z = 594 - 600, showing the ENPP1 peptides. MS survey scan contains light masses from CTC-ITB-01 and the 13C6 labeled peptides of MCF-7 at m/z = 595.33 [M + 3 H]3+ and m/z = 597.33 [M + 3 H]3+, respectively.

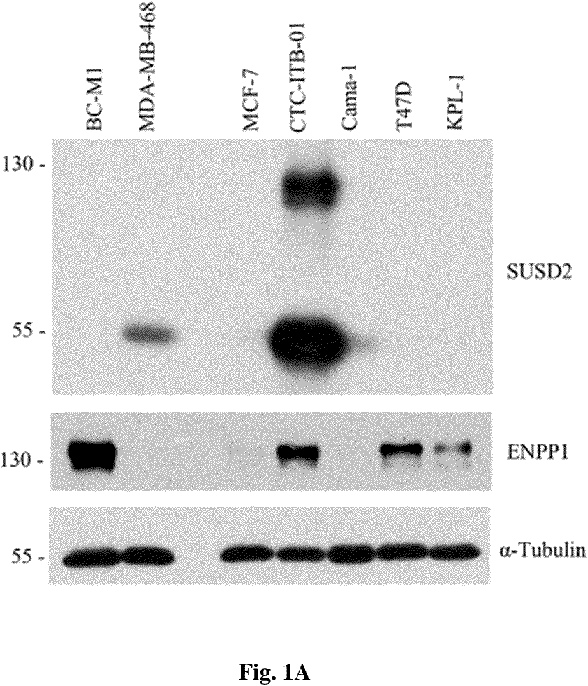

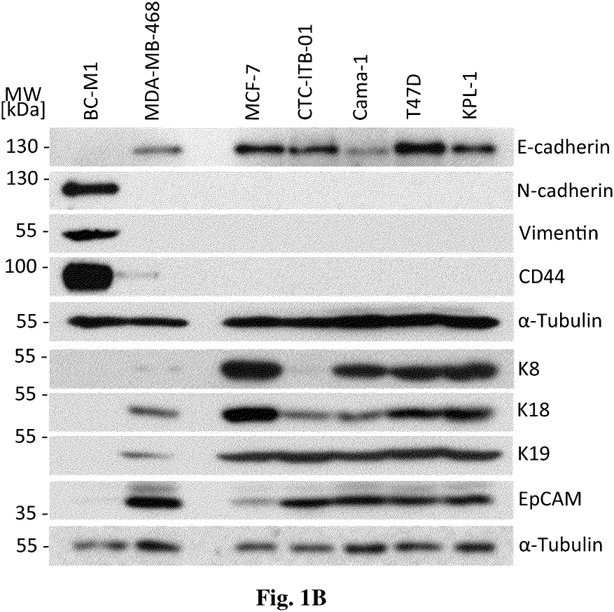

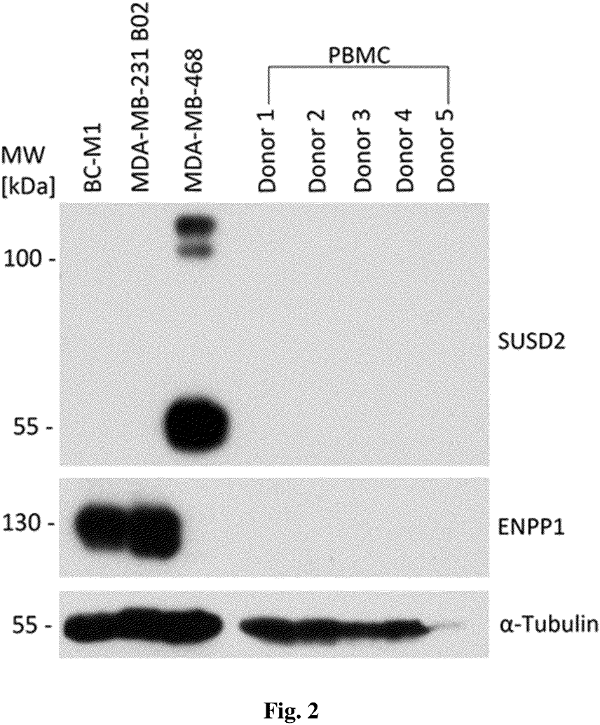

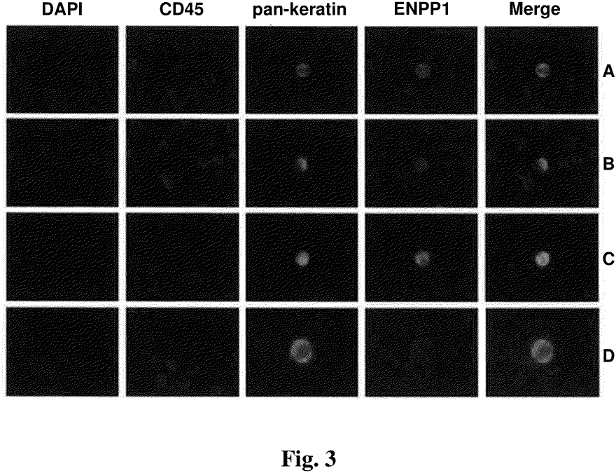

### DETAILED DESCRIPTION OF THE INVENTION

“Epithelial-to-mesenchymal transition” EMT is a normal process in embryonic development, organogenesis, gastrulation and development of the peripheral nervous system which causes epithelia-mesenchymal plasticity and enables these cells to migrate and form different tissues and organs. The process of EMT plays a relevant role in wound healing and tissue regeneration. Cancer cells from the primary tumor also take advantage of this program to start invasion and migration. As a consequence, metastases occur which are primarily responsible for cancer-related deaths. Motility and invasiveness are essential requirements for metastatic spread of malignant cells.

EMT describes a process by which tumor cells can acquire mesenchymal attributes thereby gain attributes like increased migratory capacity. Alternatively, tumors may originate from cancer stem cells. In breast cancer, cancer stem cells are malignant transformed (adult) breast stem cells. Cancer stem cells retain limitless replication potential and high migratory capacity rendering ideal candidates for the formation of distant metastasis. In addition, cancer stem cells are able to undergo uneven cell division. That means, after cell division one daughter cell is again a cancer stem cell and the other daughter cell can begin cellular differentiation. During the differentiation, the descendants gradually increase epithelial attributes, with the upregulation of epithelial marker proteins like keratins. Cancer stem cells are tumor cells with strong manifestation of a mesenchymal phenotype and very low expression of epithelial proteins like EpCam or keratins. Interestingly, the protein expression profile of cancer stem cells and EMT passed cells is very similar (see below).

Before detaching from primary tumor, the number of cell-cell contacts of the tumor cells decrease by downregulation of proteins responsible for adherence which in turn results in the loss of the apical-basal polarity. After numerous genetic changes the tumor cells are released from the primary tumor, penetrate the basement membrane and intravasate actively or passively the blood circulation or the lymphatic vessels. The CTCs are exposed to physical stress like shear forces in the blood stream or collision with blood cells. Additionally, they must avoid anoikis, a process which normally leads to apoptosis by the lack of cell-ECM contacts and escape the immune system. After reaching a distant organ, tumor cells extravasate the blood circulation by binding to the endothelium after reaching small capillaries. After extravasation, DTCs die, remain in tumor dormancy or resume proliferation to found the outgrowth of macrometastasis. In breast cancer the outgrowth of metastasis can take several years after removal of the primary tumor. One reason for the late metastatic relapse is the dormancy state of DTCs with low proliferation rates resulting in weak therapy effects in this period followed by resuming signal-triggered proliferation. CTCs and DTCs with mesenchymal attributes or after passing through EMT have advantages in motility but not in proliferation. In general, after complete EMT, the expression of epithelial proteins like EpCAM or E-Cadherin is downregulated due to the loss of cell-cell connections, cytoskeletal alterations and changes in keratin expression patterns followed by the final upregulation of mesenchymal proteins like Vimentin and N-Cadherin.

ENPP1 is a type II transmembrane glycoprotein, hydrolyzing pyrophosphate or phosphodiester bonds and negatively regulating bone mineralization (see, for example, [11], [12]; UniProtKB P22413-1). In case of single-pass type II transmembrane proteins, the C-terminus is on the extracellular side, whereas for type I transmembrane proteins like SUSD2 the N-terminus is on the extracellular side. Furthermore, ENPP1 inhibits insulin receptor activity and thus is associated with type 2 diabetes. An upregulation of ENPP1 in breast cancer specimen, and a cancer stem cell-promoting effect, mediated by an induction of the side population fraction, has been observed. Furthermore, ENPP1 facilitates drug resistance by upregulation of the ABC transporter ABCG2 and provides a tumor seeding ability. Also in lung cancer and glioblastoma, ENNP1 was associated with stem cell properties and EMT phenotype.

ENPP1 has an extracellular domain, consisting of 828 amino acids, providing the possibility to isolate or to target CTCs by antibodies against the extracellular domain of ENPP1. No expression of ENPP1 was observed in PBMCs, allowing a specific isolation of CTCs from blood, without unspecifically targeting PBMCs.

Western blot analyses revealed that ENPP1 is predominantly expressed by breast cancer cell lines with mesenchymal characteristics (MDA-MB-231, MDA-MB-231 SA, MDA-MB-231 B02, BC-M1 and BT-549) and only weakly positive in few breast cancer cell lines with epithelial characteristics (MCF-7, KPL-1, T47D). Western blot results for MCF-7, MDA-MB-231 and MDA-MB-468 are in accordance with previous described data obtained by mRNA and protein analysis. Hence, an isolation of CTCs using an antibody against ENPP1 might enrich CTCs that have undergone EMT, and which can be missed by currently applied CTC enrichment technologies, like the CellSearch® system.

The majority of bone metastases in breast cancer are osteolytic, where the normal bone is destroyed by osteoclasts. ENPP1 hydrolyzes nucleotide triphosphates to nucleoside monophosphates and diphosphates (e.g., pyrophosphate). Pyrophosphate again inhibits bone mineralization by binding to hydroxyapatite, wherefore ENPP1 could play a role in osteolytic metastasis. This assumption is in accordance with the high expression of ENPP1 by breast cancer cell lines with ability to form bone metastasis in mice (MDA-MB-231, MDA-MB-231-SA and MDA-MB-231-B02) and in BC-M1 which was established from the bone marrow of a breast cancer patient. Since MDA-MB-231 B02 and MDA-MB-231 SA have the predilection for dissemination to bone in mice compared to the parental MDA-MB-231 an upregulation of ENPP1 was expected in both sublines, if ENPP1 was essential for bone metastasis formation.

Molecular analyses of ENPP1-positive CTCs could provide deeper insights in the role of ENPP1 in tumor cell dissemination. For the isolation or inhibition of CTCs using ENPP1 as target, an antibody against the extracellular domain of ENPP1 is required. Especially, due to the ENPP1 mediated upregulation of ABC transporter, targeting of ENPP1-expressing CTCs could eliminate tumor cells responsible for therapy failure. Furthermore, if the association of ENPP1 with a stem cell-like population also applies in CTCs, ENPP1 could be used as biomarker to identify the disease driving subpopulation of CTCs.

Currently there are no publications describing the role of ENPP1 in prostate cancer. Western Blot analyses of 4 prostate cancer cell lines revealed the expression of ENPP1 in LNCap, PC-3 and PC-E1. PC-3 and PC-E1 are cell lines obtained from bone metastasis and which have a high metastatic potential.

SUSD2 is a type I transmembrane protein, comprising 822 amino acids with an extracellular domain of 758 amino acids (see, for example, [12]; UniprotKB Q9UGT4-1). The protein consists of a Sushi, von Willebrand factor type D, AMOP and a Somatomedin B domain. Amino acids 1 to 27 represent the signal peptide and the protein is cleaved at the GDPH sequence. Furthermore, the protein has several predicted N-linked glycosylation sites. SUSD2 was chosen as a promising target for CTC isolation due to the extracellular domain and its absence in PBMCs, proven by Western blot analysis. SUSD2 was previously described as marker for isolation of mesenchymal stem cells in human palatine tonsil, bone marrow and endometrium. Furthermore, it was shown to play a role in neuronal development. A tumor related role was first described for the mouse homolog of SUSD2, where a tumor-reversing effect was found. The role of human SUSD2 was investigated in cancer, showing a tumor-promoting impact in breast cancer and tumor inhibiting effects in renal cell carcinoma, lung cancer and high grade serous ovarian cancer. IHC analysis of matched sets of breast cancer and normal breast tissues revealed high SUSD2 expression in all stages of breast cancer, while in benign breast tissue expression was only observed in endothelial cells lining the blood vessels and capillaries. SUSD2 was associated with increased invasion through Matrigel, suggesting a metastasis-promoting effect. An angiogenesis mediating impact of SUSD2 was shown to be conveyed by recruitment of tumor associated macrophages via the chemoattractant MCP-1.

The theoretical mass of SUSD2, considering the complete amino acid sequence is 90.2 kDa. Subtracting the mass of the signal peptide, a mass of 87.5 kDa is predicted for the mature SUSD2 protein. However, the Western blots showed a dominant band at 55 kDa and another band with lower intensity between 110 - 130 kDa, depending on the cell line. Since at least four N-linked glycosylation sites are described for SUSD2 it was assumed that the band at 110 - 130 kDa is a glycosylated form of the full-length protein. The identity of the 55 kDa band was proven by LC-MS/MS analysis in this work, providing eight unique peptides of SUSD2. Thus, the 55 kDa signal detected by Western blot analysis is most likely specific for SUSD2, suggesting posttranslational cleavage or translation at an alternative initiation site. One of the eight identified SUSD2 peptides found at 55 kDa starts with the amino acid methionine and is located at the N-terminal side of the SUSD2 peptide. As the genetic code for methionine, AUG, also serves as an initiation site for the mRNA translation, a shorter version of SUSD2 might have been generated due to the use of an alternative start codon. However, if translation started at this methionine (amino acid 85) and ended at the stop codon ATG, which is located behind the base code for proline 822, a mass of 81.4 kDa is predicted. Thus, the 55 kDa form of SUSD2 is more likely a cleavage product generated after translation of the complete protein. Nevertheless, translation at an alternative initiation site might still have happened yielding in a signal detected between 110 - 130 kDa after glycosylation.

One of many hurdles for a CTC to survive in the bloodstream is the immune system. Several mechanisms are described for tumor cells to evade the immune system, including abnormal expression of major histocompatibility complex class I proteins, loss and modification of tumor associated antigens or blocking the antitumor function of T-cells. The latter can be mediated by galectin-1, which induces apoptosis in T-cells. An interaction of SUSD2 and galectin-1 was described, where SUSD2 facilitates the cell surface presentation of galectin-1. CTC-ITB-01 is strongly SUSD2-positive and might possess immune system evading mechanisms as this cell line originates from CTCs that survived in the bloodstream of a breast cancer patient. However, this attribute was most likely not mediated by galectin-1 since only a very weak galectin-1 expression was observed in CTC-ITB-01.

The cancer stem cell theory includes the hypothesis that cancer stem cells might originate from normal stem cells. The presence of mammary stem cells is supported by the changes occurring in the mammary gland after pregnancy and lactation and in accordance with the cancer stem cell theory, transformation of the mammary stem cells could initiate breast cancer. Since SUSD2 is associated with mesenchymal stem cell-like cells, this protein might further support the stem cell-like properties of CTC-ITB-01 and suggests that the cell line might originated from a mammary stem cell. Prove of this theory requires evidence for SUSD2 expression on mammary stem cells.

The strikingly high expression of SUSD2 in CTC-ITB-01 and the promising findings in CTC-ITB-01 MIND, indicated that SUSD2 might be a suitable target for CTC enrichment. Furthermore, the presence of SUSD2 in cell lines of different phenotypes could enable to target a broad range of different CTC subtypes (epithelial, hybrid epithelial/mesenchymal and mesenchymal). As mentioned above, SUSD2 was utilized as marker for the isolation of mesenchymal stem cell-like cells using FACS or MACS®. To enrich SUSD2-expressing cells by MACS®, these studies applied an indirect system by using PE-conjugated antibodies against SUSD2 and anti-PE MicroBeads.

Referring to SUSD2, the present inventors showed that separation of SUSD2-positive CTCs can be achieved using the MACS® system together with SUSD2 MicroBeads. Customized human SUSD2 MicroBeads were produced by Miltenyi Biotec, where the anti-SUSD2 antibody is directly coupled to the superparamagnetic beads. The system was first tested by spiking experiments using the highly SUSD2-positive CTC-ITB-01 and the SUSD2-negative T47D cell lines. Suitability of both cell lines as positive and negative control was proven by FACS analysis, to ensure that the obtained recovery rates are not influenced by a missing or present expression of SUSD2 on the positive or negative control, respectively. FACS analysis showed that 100 % of adherent and 98 % of non-adherent CTC-ITB-01 cells were positive for SUSD2, while only 0.98 % of T47D cells were SUSD2-positive, proving the suitability of these controls. The MACS® spiking experiments yielded in recovery rates of 70.67 % and 3.33 % for CTC-ITB-01 and T47D, respectively, while 4.67 % of CTC-ITB-01 and 64.00 % T47D cells were detected in the combined flow-through and wash fraction. These results indicated a specific separation of SUSD2-positive cells with the SUSD2 MicroBeads.

CTC-ITB-01 cells found in the elution fraction showed a strong circular mouse IgG staining in IF analysis, proving the binding of the SUSD2 MicroBeads to the SUSD2-expressing CTC-ITB-01 cells. Hence, the specific binding of SUSD2 MicroBeads to SUSD2-positive cells was proven. IF analysis of CTC-ITB-01 cells in the combined flow-through and wash fraction showed at most a very weak staining for mouse IgG, suggesting that those cells were not bound by the SUSD2 MicroBeads or in much lower extent compared to those cells detected in the elution fraction. Even though probably all CTC-ITB-01 cells express SUSD2, some cells present lower amount of SUSD2 proteins on their surface, as heterogeneous SUSD2 expression was observed during IF analysis of CTC-ITB-01. The CTC-ITB-01 cells with lower SUSD2 expression provide fewer binding sites for the SUSD2 MicroBeads and were probably therefore not retained in the magnetic field. Furthermore, the accessibility of CTC-ITB-01 cells to the SUSD2 MicroBeads might have been lowered, if cells were surrounded by many PBMCs.

Subjecting blood from patients with breast cancer to the MACS® system SUSD2-positive CTCs were enriched in the elution fraction for 3 patients, but also SUSD2-negative CTCs were observed in the same fraction. Furthermore, some SUSD2-positive CTCs were observed in the combined flow-through and wash fraction for one patient with very high CTC count. This could be due to the fact that the system was optimized for a low number of 50 tumor cells during spiking experiments, as the CTC frequency is usually low (one tumor cell in a background of 10⁶-10⁷ blood cells). But this patient had an uncommonly high number of CTCs, which likely requires a different protocol with higher amount of SUSD2 MicroBeads to label all SUSD2-positive cells with the MicroBeads. This could decrease the appearance of SUSD2-positive cells in the combined flow-through and wash fraction. Due to the very high number of CTCs, SUSD2-negative cells appeared as background in the elution fraction, which is in accordance with the observation that unlabeled leukocytes are also found in the elution fraction. Due to the high number, leukocytes are not completely removed during the washing step and leave the column during the elution step, appearing together with the labeled cells. Likely, a more intense washing step would have yielded in lower number of SUSD2-negative cells in the elution fraction.

To provide further evidence for SUSD2 expression on CTCs, the CellSearch® system was used. Therefore, the anti-SUSD2-Vio Bright FITC antibody was tested in the CellSearch® system by spiking experiments using CTC-ITB-01 and MDA-MB-468 as positive controls and T47D as negative control. Since results were in accordance with previous obtained data by Western blot and IF analysis, the antibody was proven to be suitable for SUSD2 detection in CellSearch® enriched tumor cells. Of 23 CTC-positive patients, 9 patients harbored at least one SUSD2-positive CTC. These 9 patients showed a heterogenous distribution in the amount of SUSD2-positive CTCs ranging from 0.39 % to 60 %. The majority of the SUSD2-positive CTCs showed low SUSD2 expression. Two blood samples each were analyzed for four patients. Two of those patients showed SUSD2-positive CTCs only in one blood sample. This might be due to a low specificity of the anti-SUSD2 Vio Bright FITC antibody or the low frequency of SUSD2-positive CTCs in those patients. However, the suitability of the anti-SUSD2-Vio Bright FITC antibody in the CellSearch® system was proven in spiking experiments and for the other two patients SUSD2-positive CTCs were detected in both blood samples, further indicating a sufficient specificity of the anti-SUSD2 Vio Bright FITC antibody.

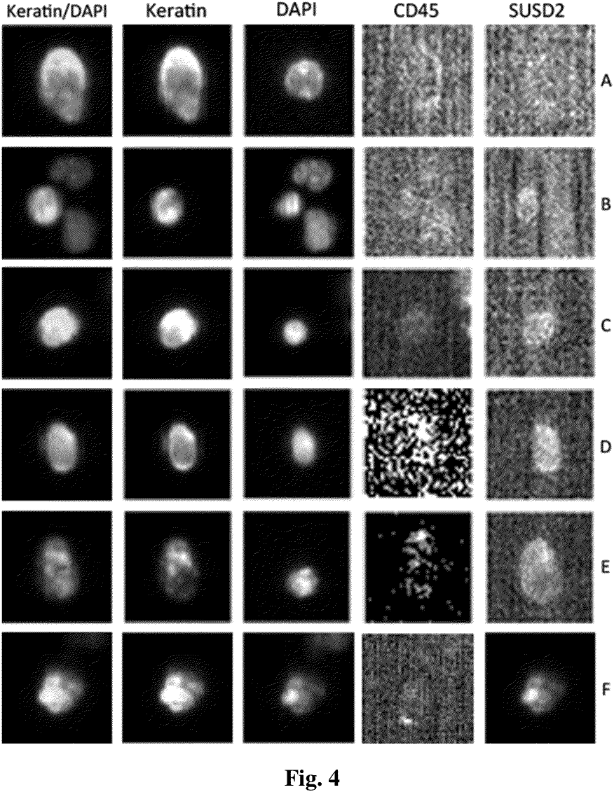

Evidence for expression of SUSD2 and ENPP1 on patient CTCs was provided by CellSearch analyses and immunofluorescence analyses, respectively (FIGS. 3-4, 10). FIG. 3 shows detection of ENPP1 on CTCs of patients with breast cancer. Cells were stained with anti-ENPP1 antibody on a glass slide. Furthermore, pan-keratin was used as tumor cell marker, CD45 as leukocyte marker and nuclei were visualized using DAPI. A: ENPP1-positive CTC of patient 1. B: ENPP1-negative CTC of patient 1. C: ENPP1 - positive CTC of patient 2. D: ENPPl-negative CTC of patient 2. Images were taken at 400 x magnification. FIG. 4 shows detection of SUSD2 expression on CTCs. Representative pictures of CTCs enriched from blood of patients with metastatic breast cancer using the CellSearch ® system are shown. CTCs were identified with the CellSearch® CTC Kit as keratin+ /DAPI+ /CD45- cells. SUSD2 expression was investigated with the anti-SUSD2 Vio Bright-FITC antibody. A: SUSD2-negative CTC. B and C: CTCs with low SUSD2 expression. D and E: CTCs with moderate SUSD2 expression. F: CTC with strong SUSD2 expression.

Therefore, these proteins can be used as biomarkers for CTC detection and characterization. Since SUSD2 and ENPP1 are transmembrane proteins, enrichment of CTCs targeting these proteins is possible. Therefore, SUSD2 was targeted for CTC enrichment in this invention.

Since SUSD2 and ENPP1 are transmembrane proteins with large extracellular domain, these extracellular regions are directly accessible for anti-SUSD2 and anti-ENPP1 antibodies without further manipulation of the cells (e. g. permeabilization). This allows binding of anti-SUSD2 and anti-ENPP1 antibodies to the extracellular domains, in which the antibodies are coupled to magnetic beads (MACS, see below). By this approach, it is possible to isolate viable CTC (or DTC) from the blood of cancer patients. Unlike other approaches, MACS can be up scaled to larger volumes allowing the isolation of CTC from larger blood volumes. This increases the possibility to isolate tumor cells from the sample so that the number of CTC positive blood samples of breast cancer patients can be increased.

In addition, the CTC isolation by MACS is a very mild method, which allows the subsequent analysis of the isolated intact cells by other methods like DNA mutation analysis. Alternatively, since the cells isolated by MACS are still viable, these cells can be cultured allowing further analyses of their genotypic and phenotypic characteristics.

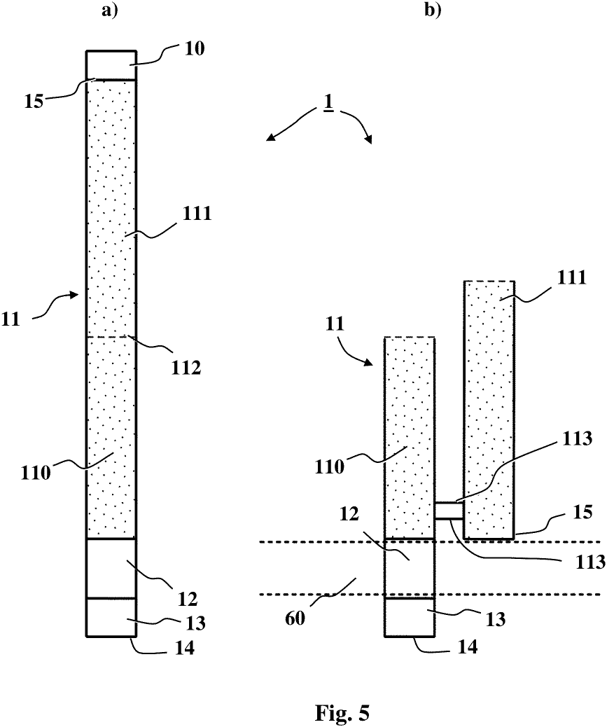

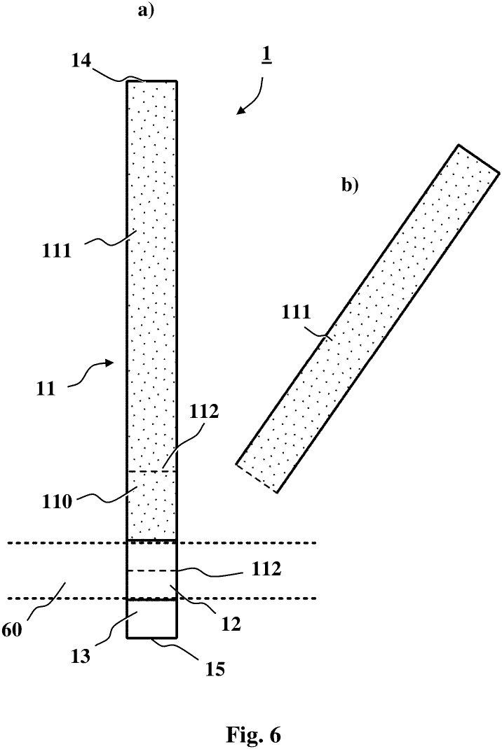

In FIGS. 5 and 6, the structures of the transmembrane proteins SUSD2 or ENPP1 (both denoted with reference numeral 1) are schematically depicted, respectively. SUSD2 is anchored in the cell membrane 60 via a transmembrane domain 12 (amino acids 786-806). The N-terminal 15 is located extracellularly, the C-terminal 14 is located at the end of the intracellular (cytoplasmic) domain 13. The extracellular domain 11 of SUSD2 has a GDPH cleavage site 112, such that the extracellular domain 11 can be split into a first truncated membrane-bound extracellular domain fragment 11 and a second fragment 111 which is bound to the first fragment 110 via disulfide bridge (see FIG. 5b). The extracellular domain 11 can, for example, be a target for MACS. Both the extracellular domain 11 and the cytoplasmic domain 13 can be a target for immunofluorescence detection, for example. ENPP1 is also anchored in the cell membrane 60 via a transmembrane domain 12 (amino acids 77-97). In contrast to SUSD2, the C-terminal 14 of ENPP1 is located extracellularly, whereas the N-terminal 15 is located at the end of the intracellular (cytoplasmic) domain 13. The extracellular domain 11 has potentially two cleavage sites 112. ENPP1 is likely cleaved intracellularly such that a soluble fragment 111 can be secreted (see FIG. 6b). The extracellular domain 11 can, for example, be a target for MACS. Both the extracellular domain 11 and the cytoplasmic domain 13 can be a target for immunofluorescence detection, for example. The soluble protein fragment 111 can be a target for an ELISA.

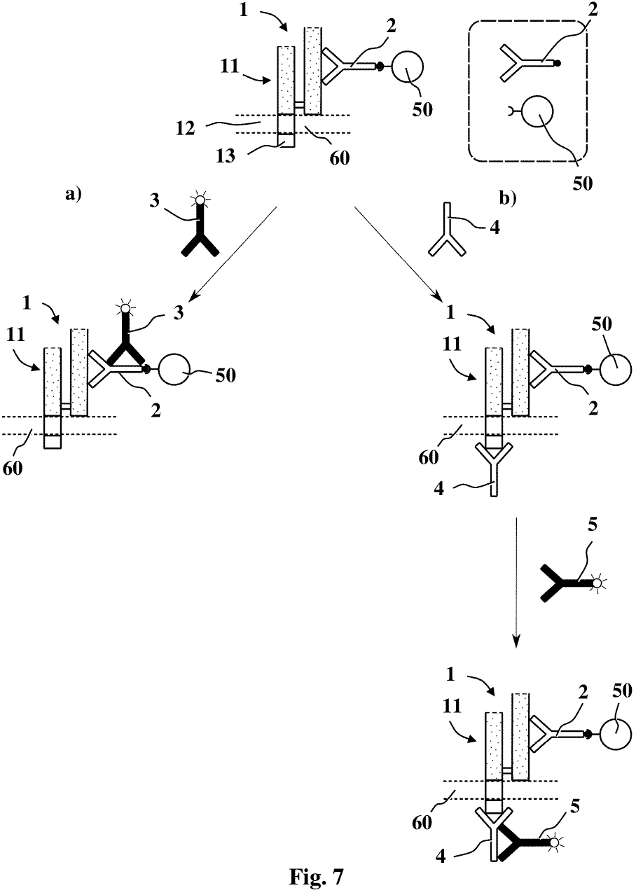

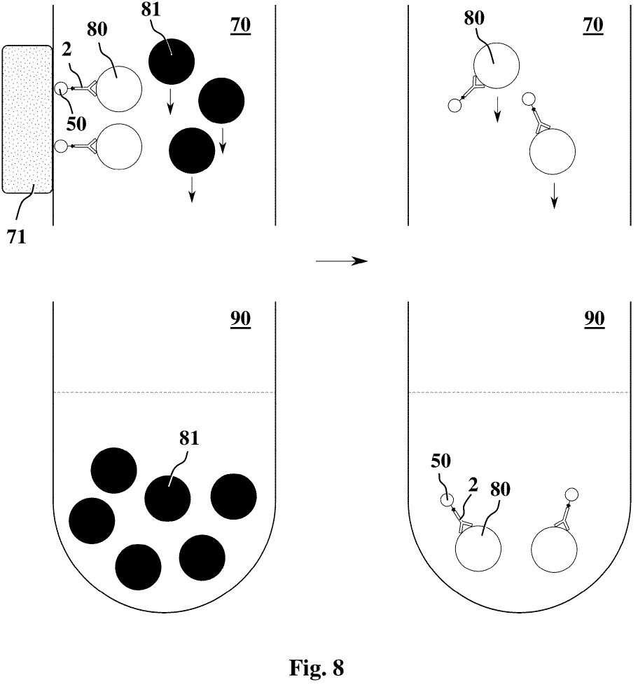

As one example, the method of the invention can be carried out using magnetic-activated cell sorting (MACS®), using magnetic-activated anti-SUSD2 or anti-ENPP1 antibodies, i.e., anti-SUSD2 or anti-ENPP1 antibodies coupled to magnetic nanoparticles, as described below in more detail in relation to FIGS. 7, 8. However, isolation of CTC and/or DTC can be achieved via an anti-SUSD2 or anti-ENPP1 antibody coupled to any suitable substrate or functionalized on any surface.

FIG. 7 shows an example of an embodiment of the method of the invention using magnetic activated cell sorting (MACS®) for the isolation of SUSD2⁺ or ENPP1⁺ cells, i.e., SUSD2-positive or ENPP1-positive cells. SUSD2⁺ or ENPP1⁺ cells are here represented by SUSD2 or ENPP1 1 anchored in the cell-membrane 60. In a first step, a CTC or DTC, carrying SUSD2 or ENPP1 on its cell-surface, is captured with a first primary antibody 2 coupled to the magnetic nanoparticle 50 which is directed to the extracellular domain 11 of SUSD2 or ENPP1 1. The first primary antibody 2 is preferably a polyclonal antibody, particularly preferred a (polyclonal) antibody binding to the part of the extracellular domain 11 of SUSD2 or ENPP1 that is not cleaved off, i.e., binding also to the truncated version of SUSD2 or ENPP1. SUSD2⁺ or ENPP1⁺ CTC/DTCs can be isolated via MACS®, as described in relation to FIG. 8. It should, however, be noted, that the isolation can be performed using a modified CellSearch system (with SUSD2 or ENPP1 instead of EpCAM as a marker) or other isolation procedures, e.g., via anti- SUSD2 or anti-ENPP1 antibodies bound to a solid phase other than a magnetic nanoparticle 50. For the subsequent detection of CTC/DTCs, two approaches are shown. In a first approach (see FIG. 7a) a fluorescently labeled first secondary antibody 3 directed against the first primary anti-SUSD2 antibody or anti-ENPP1 antibody 2, or against the complex composed of the first primary anti-SUSD2 antibody or anti-ENPP1 antibody 2, the magnetic nanoparticle 50 and the components coupling the magnetic nanoparticle 50 to the first primary anti-SUSD2 antibody or anti-ENPP1 body 2, (e.g. biotin and streptavidin), is added in order to detect SUSD2-positive or ENPP1-positive CTC/DTCs. In another approach (see FIG. 7b), a second primary anti-SUSD2 antibody or anti-ENPP1 body 4, directed against a preferably cytoplasmic terminal region of the transmembrane protein 1, i.e. a preferably C-terminal region in case of SUSD2 and a preferably N-terminal region in case of ENPP1, of the intracellular (cytoplasmic) domain 13 of SUSD2 or ENPP1 1, is added and bound to SUSD2 or ENPP1 1, and a fluorescently labeled second secondary antibody 5 directed against the second primary anti-SUSD2 antibody or anti-ENPP1 body 4 is added in order to detect SUSD2-positive or ENPP1-positive CTC/DTCs.

The method of the invention can be adapted to various geometries and topologies. First, the coupling of an anti-SUSD2 antibody or anti-ENPP1 body is not limited to coupling to magnetic nanoparticles, as described above in relation to FIG. 6. Rather, an anti-SUSD2 antibody or anti-ENPP1 body can be coupled by different methods to any suitable material, e.g. any solid phase material used in the prior art for immobilizing antibodies. Such coupling methods may be for example the application of reactive cosslinkers like NHS esters (N-hydroxysuccinimide esters) and imidoesters, which allow the coupling of anti-SUSD2 bodies or anti-ENPP1 bodies to a suitable material or surface. Another option is the covalent conjugation of the anti-SUSD2 antibody or anti-ENPP1 body to fluorescent (non-magnetic or magnetic) nanoparticles. This approach could be used for detection (fluorescent nanoparticles) or isolation/enrichment (magnetic nanoparticles). Coupling of an anti-SUSD2 antibody or anti-ENPP1 body to a fluorescent magnetic nanoparticle is advantageous in that this direct approach would work without a secondary antibody reaction. As already mentioned, different materials/surfaces instead of magnetic nanoparticles to which the anti-SUSD2 antibody or anti-ENPP1 body is coupled can be applied. Such material may be for example Sepharose® (a crosslinked, beaded-form of agarose, or nanoparticles (fluorescence labeled or unlabeled), but other materials or surfaces may also be applied. For example, the cell collector (see U.S. Pat. US10022109B2) can be adjusted to the capture of SUSD1- or ENPP1-positive CTCs or DTCs by functionalizing the device with anti-SUSD2 antibody or anti-ENPP1 body. Another example is the detection/isolation by a modified commercial system called “CellSearch”, which is an immunomagnetic technology that uses anti-EpCAM antibodies coupled to ferrofluid nanoparticles for separation of EpCAM-expressing cells. Supplement or substitution of anti-EpCAM antibodies with anti-SUSD2 antibody or anti-ENPP1 body coupled to ferrofluid nanoparticles in the CellSearch approach enriches SUSD2-positive or ENPP1-positive CTC or improves the number of enriched CTC, respectively.

Currently, immunomagnetic methods (MACS) are widely-used to isolate tumor cells. For example, the immunomagnetic isolation via the extracellular domain of EpCAM is used to capture EpCAM-positive CTCs and DTCs in metastatic breast cancer. This approach enables the isolation of CTCs or DTCs with an epithelial phenotype. The disadvantage of this approach is the loss of EpCAM-negative CTCs and DTCs. EpCAM-negative tumor cells might have undergone a complete EMT which leads to a mesenchymal phenotype or a partial EMT which leads to a hybrid epithelial/mesenchymal phenotype. Mesenchymal CTCs are thought to be more invasive than the epithelial counterparts but the hybrid epithelial/mesenchymal phenotype is linked with metastasis formation and metastatic relapse. These findings indicate that there is need for a cell surface molecule which can be used for isolating CTCs or DTCs, for example a MACS-based method for the isolation of CTCs with mesenchymal or hybrid epithelial/mesenchymal attributes. The inventors have found that the large extracellular domain of SUSD2 and ENPP1 is applicable for, for example, MACS-based isolation of CTCs/DTCs with different phenotype (epithelial, mesenchymal or hybrid epithelial/mesenchymal). Due to the fact that different biological variants of SUSD2 and ENPP1 correlate with dissemination and invasion of tumor cells, SUSD2-positive and ENPP1-positive tumor cells might provide information about metastasis formation-potential and metastasis development.

For the labeling procedure an indirect and a direct variant can be distinguished. The antigen of interest on the cell surface can be hybridized with an antibody or a biotinylated antibody followed by the binding of the super-paramagnetic nanoparticles (MACS-nanobeads, size range: 20-100 nm) which are conjugated to a secondary antibody (recognizing the primary antibody) or to Streptavidin (recognizing the biotinylated antibody). This approach represents the indirect technique. The faster direct approach is represented by the direct binding of MACS bead-conjugated antibodies to the cell surface antigen of interest. For the isolation procedure a positive and a negative selection can be differentiated. The positive selection enriches the cells harboring the antigen of interest on their cell surface and the cells lacking the cell surface antigen are discarded. For this purpose, the antigen-positive cells are captured with an antibody or other binding agent coupled to a magnetic nanoparticle and retained by a magnetic field. On the other hand, the negative selection isolates all cells lacking the cell surface antigen of interest by capturing them with a binding agent, e.g., antibody, linked to a magnetic nanoparticle, while the cells expressing the cell surface antigen are not captured and released from, for example, a MACS column. After successful labeling with magnetic nanoparticles the cells are applied to a ferromagnetic iron-column positioned in a magnetic field. In FIG. 8, the procedure is schematically shown for the positive selection of cells by a cell surface molecule-dependent MACS isolation procedure. Cells 80 are labeled with a biotinylated antibody 2 coupled via streptavidin to a magnetic nanoparticle 50. The antibody 2 is directed against a membrane-bound antigen, e.g. a membrane-bound protein having an extracellular domain, like SUSD2 or ENPP1. Nanoparticle-labeled cells 80 retain in the column 70 by the application of a magnetic field of a magnet 71 and unlabeled or antigen-negative cells 81 pass the column 70 and will be discarded, or collected in a collecting vessel 90. Thereafter (see FIG. 8b), the magnetic column 70 is removed from the magnet 71, the labeled cells 80 are eluted and collected in a collecting vessel 90, and further analysis can be performed. The complex composed of the biotinylated antibody 2 and the Streptavidin-conjugated magnetic nanoparticle 50 recognizes and binds to the target cell 80. The cell-antibody-nanoparticle complex binds to the MACS column 70 placed into a permanent magnet 71. The unlabeled cells 81 or cells negative for the surface protein are discarded after passing the column 70 without binding. After removal of the column 70 from the magnet 71 the labeled cells 80 are collected and represent an enriched population.

In principle, it is possible to carry out the isolation of CTCs or DTCs with only the anti-SUSD2 antibody or anti-ENPP1 body coupled to the nanoparticles (or similar suitable substrates). The identity of the isolated cells can be confirmed, for example, by immunofluorescence by applying a second anti-SUSD2 antibody or anti-ENPP1 body and a fluorescently labeled secondary antibody directed against the second anti-SUSD2 antibody or anti-ENPP1 body, or a fluorescently labeled secondary antibody directed against the first SUSD2-antibody or ENPP1-antibody (anti-catching antibody). Additionally, an anti-keratin antibody may be used in combination with the second anti-SUSD2 antibody or anti-ENPP1 body or the anti-catching antibody. Alternatively, the cells can be isolated and transferred to another vessel for subsequent procedures. Such procedures may include a cell lysis step followed by whole genome amplification followed by DNA sequencing of the genome to identify tumor-relevant gene mutations. Such gene mutations might be responsible for therapy resistance if a gene of a therapeutic target is mutated (e. g. EGFR). An alternative evolving approach is the analysis of the isolated cells on proteome level, for example by single cell mass spectrometry. In that case cancer relevant target structures (most therapy targets are proteins) can be directly assessed, which helps the prediction of therapy success of the patients. Depending on the isolation procedure, isolated cells are viable and thus can be used for subsequent functional in vitro (e.g., migration and invasion assay) and in vivo (e.g., patient-derived xenograft in zebrafish embryos) assays possibly after expansion in long-term cultures.

As an alternative to magnetic beads (nanoparticles), the anti-SUSD2 antibody or anti-ENPP1 antibody can be coupled to an organic matrix such as Sepharose®, and this can be added to the sample. Sepharose forms a kind of slurry. When you have the complex CTC-anti-SUSD2 antibody-Sepharose or CTC-anti-ENPP1 body-Sepharose in the sample, you can simply let the complex settle down (no centrifugation step necessary) and discard the supernatant. If you want to remove normal cells that got stuck in the slurry, simply dissolve the slurry in a suitable buffer and let it settle down again and discard the supernatant. Accordingly, the invention can be carried out with an organic matrix (e. g. Sepharose) having anti-SUSD2 antibody or anti-ENPP1 antibody coupled via reactive functional groups, wherein the separation involves gravitation force, for example by settlement of the CTC-anti-SUSD2 antibody-Sepharose or CTC-anti-ENPP1 body-Sepharose complex or centrifugation.

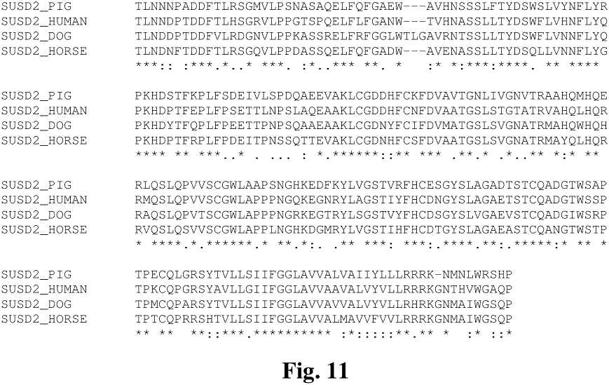

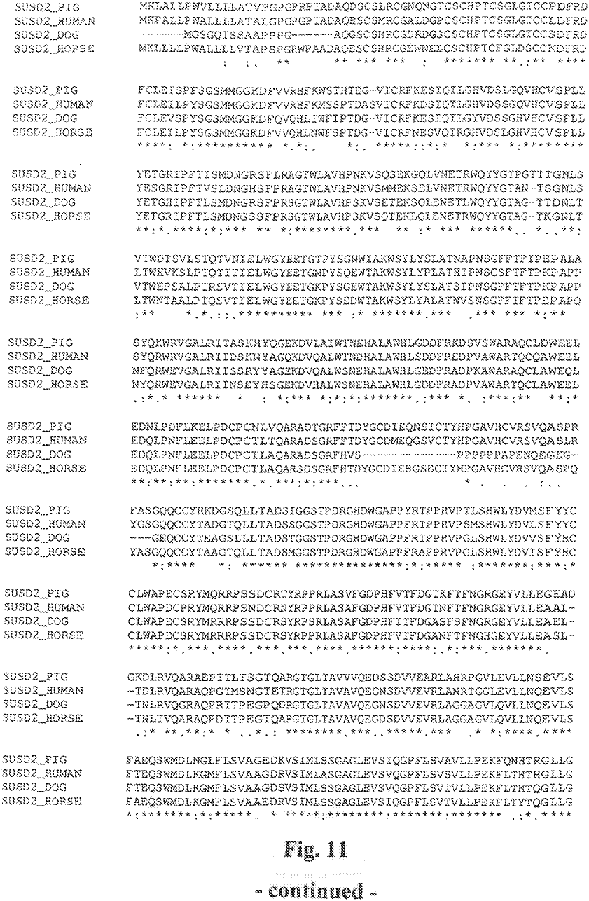

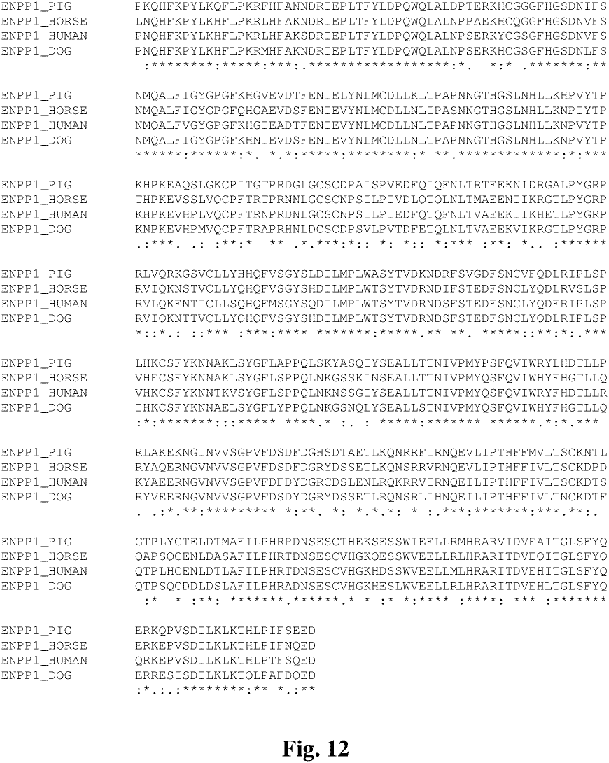

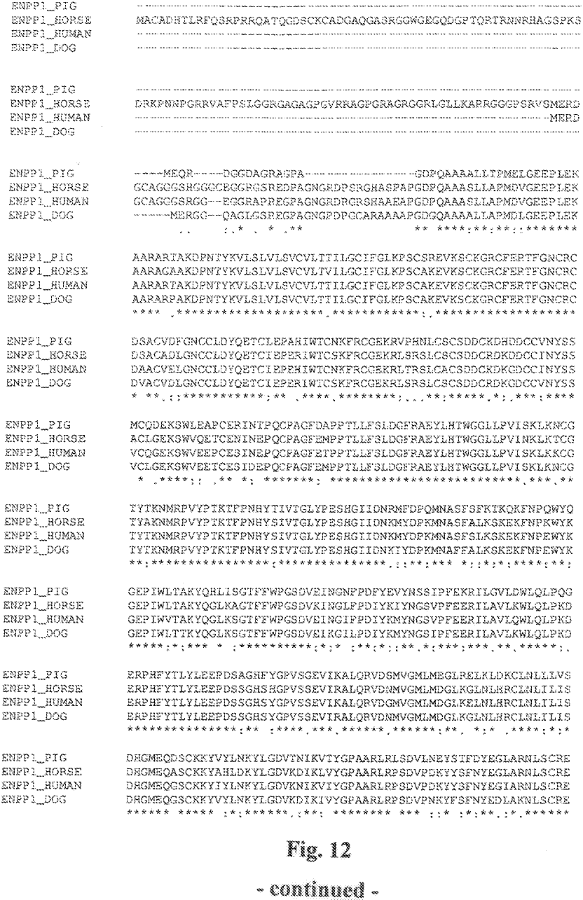

Further, the distribution of SUSD2 and ENPP1 was investigated in other mammals by database search (FIG. 11, FIG. 12). After identification of the corresponding proteins in dog, horse and pig by sequence homology search, we performed a sequence homology analysis. SUSD2 and ENPP1 are present in these animals with a sequence homology of more than 75%.

The present invention thus uses the Sushi-domain-containing protein 2 (SUSD2) and Ectonucleotide pyrophosphatase/phosphodiesterase family member 1 (ENPP1) as a biomarker for the isolation, preferably the isolation and detection of CTCs or DTCs having an epithelial phenotype (eCTC, eDTC), a mesenchymal phenotype (mCTC, mDTC) or a hybrid epithelial/mesenchymal phenotype (emCTC, emDTC), preferably a mesenchymal or hybrid epithelial/mesenchymal phenotype. It has been found that these biomarkers are, for example, strongly expressed in CTCs from breast cancer. The invention can, however, also be used in the context of other cancers with a high potential to form metastases, e.g., prostate cancer, pancreatic cancer, head and neck cancer, lung cancer or malignant mesothelioma.

The term “isolation” in relation to targeted cells like CTCs or DTCs refers to the separation of these cells from a biological sample containing the cells, for example, from peripheral blood, lymph, bone marrow or the like. The term includes the separation of these cells from other, not targeted cells, e.g., the separation of SUSD2-positive and ENPP1-positive CTCs or DTCs from the biological sample medium and other cells in the same sample, e.g., SUSD2-negative or ENPPl-negative CTCs or DTCs. The term encompasses enrichment of the targeted cells, i.e., increasing the concentration of the targeted cells by removal of fractions not containing targeted cells, or removal of targeted cells to a collection medium, or by accumulating targeted cells in a part or compartment of a container or in an area of an object slide.

The term “detection” in relation to targeted cells like CTCs or DTCs refers to identification of the targeted cells in a biological sample, in a medium enriched with the targeted cells or in a medium at least mainly, preferably exclusively containing the targeted cells. Identification means suitably labeling the cells and qualitatively or quantitatively detecting the presence of the cells.

The term “subject” as used herein refers to a non-human animal, preferably a non-human mammal, for example a cat, dog, horse, cattle, or to a human.

The term “nanoparticle” as used herein relates to particles of a size of 1 to ≤100 nm. In relation to a plurality of nanoparticles the term means that the particle size of at least half of the particles in the number size distribution of the plurality of nanoparticles is 100 nm or below.

### Material and Methods

To avoid an unwanted isolation of non-tumor cells during CTC isolation by targeting a cell surface protein, an absence of this protein on PBMCs is necessary. Database search declared no expression of SUSD2 or ENPP1 by PBMCs. To approve that the selected proteins are suitable biomarkers for CTC isolation, this information was verified by Western blot analysis using PBMC lysates.

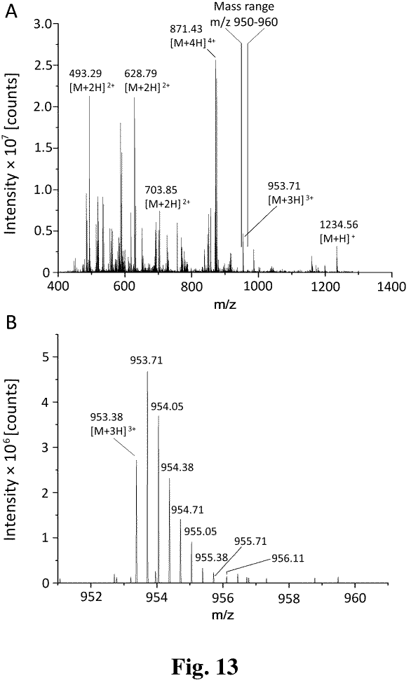

SUSD2: SUSD2 was identified in five replicates by 27 unique peptides, including peptide CGALDGPCSCHPTCSGLGTCCLDFR (FIG. 13) in a SILAC-based proteome analysis comparing the CTC-derived cell line CTC-ITB-01 and MCF-7. The monoisotopic peak of the peptide CGALDGPCSCHPTCSGLGTCCLDFR (theoretical monoisotopic mass = 2,857 \.17 Da) was observed in the MS survey scan at m/z = 953.38 [M + 3 H]^{3 +} (FIG. 13). Due to the large sequence of this peptide, a high number of isotopic peaks is present. Signals present at m/z= 955.38 and 955.71 [M + 3 H]^{3 +} might result from a corresponding heavy peptide ion, but the signal intensities suggest that these signals are rather isotopic peaks of the light peptide ion. Additionally, data of all analyzed peptides identified for SUSD2 obtained by MaxQuant software indicated no expression of SUSD2 in MCF-7, further suggesting that signals at m/z = 955.38 and 955.71 [M + 3 H]^{3 +} are isotopic peaks from the light peptide ion.

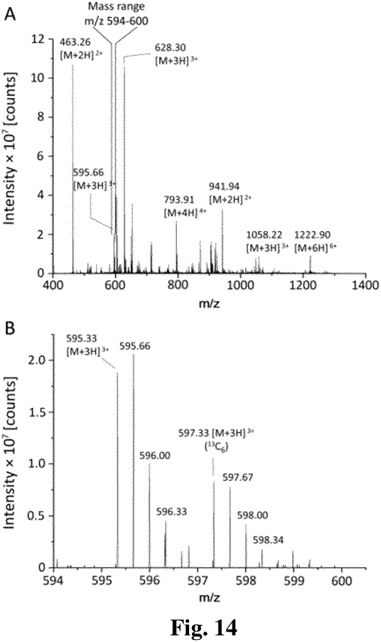

ENPP1: ENPP1 was identified in five replicates by 14 unique peptides in a SILAC-based proteome analysis comparing the CTC-derived cell line CTC-ITB-01 and MCF-7 (Table 1). ENPP1 was expressed by both cell lines, proven by the simultan presence of light and heavy peptide ion signals in the MS survey scans, e.g. observed for the ENPP1 peptide AEYLHTWGGLLPVISK (FIG. 14). For this peptide, the light peptide ion signal was observed at m/z = 595.33 [M + 3 H]³⁺, while the heavy peptide ion signal was found at m/z = 597.33 [M + 3 H]³⁺ caused by the incorporation of ¹³C₆ lysine. However, the light peptide shows higher signal intensity than the heavy peptide, indicating a higher expression of ENPP1 in CTC-ITB-01 compared to MCF-7.

TABLE 1

LC-MS/MS results of the CTC biomarker candidates Determination of differential expression of the CTC biomarker candidates SUSD2 and ENNP1, between MCF-7 and CTC-ITB-01

| Uniprot accession number | Protein short name | Total number of peptides analyzed ^{a} | Number of unique peptides analyzed ^{b} | Number of biological replicates | Average of normalized ratio (H/L) | Standard deviation | p-value ^{c} |
|---|---|---|---|---|---|---|---|
| Q9UGT4 | SUSD2 | 150 | 27 | 5 | only in CTC-ITB-01 | - | - |
| P22413 | ENPP1 | 39 | 14 | 5 | - 2.93 | 0.052 | 4.697 × 10⁻³⁶ |

^{a} Total number of all analyzed peptides identified for one protein (unique and not unique). Same peptides were counted multiple times (once per each fraction and replicate);
^{b} Number of peptides exclusively associated with one protein. Number contains only different peptides;
^{c} Student’s t-test, value of p< 0.05 was considered as significant difference

CTCs can be enriched in different ways, e.g. by density gradient centrifugation, magnetic cell separation or with the CellSearch system. Following enrichment, CTCs can be detected and characterized by immunocytochemistry. Proteins on the cell surface of CTCs can be used for both enrichment and detection. Furthermore, cell surface proteins represent potential targets for therapeutic antibodies. Thus, it was aimed to prove the presence of SUSD2 and ENPP1 on CTCs, wherefore immunofluorescence (IF) staining protocols were established. Therefore, cytospins were prepared, using healthy donor blood spiked with cancer cell line cells, to mimic a patient sample containing CTCs in the blood.

For the transmembrane proteins ENPP1 and SUSD2 an immunofluorescence staining protocol was established and successfully applied to patient slides. Thus, the IF staining protocol couldbe used in the future for the detection of CTCs in patient samples. Furthermore, a system for magnetic cell separation (MACS®) of SUSD2-positive cells was established, which can potentially be used for the isolation of SUSD2-positive CTCs from the blood of breast cancer patients. For this, evidence for a SUSD2 expression in CTCs of patients was required. Therefore, the CellSearch® system was used to enrich CTCs from the blood of patients with metastatic breast cancer. Subsequently, the enriched CTCs were analyzed for SUSD2 expression by immunofluorescence staining. SUSD2-expressing CTCs were detected in 9 of 23 CTC-positive patients.

The invention uses a column-based method, which allows magnetic separation of cells based on surface antigens (see FIG. 8). Here, SUSD2 MicroBeads (Miltenyi Biotec, Bergisch Gladbach, Germany) were used, which are 50-nm superparamagnetic particles that are conjugated to an antibody against SUSD2. This allows the enrichment of SUSD2-positive cells such as CTCs from a biological sample such as blood.

For spiking experiments, PBMCs from healthy donor blood were isolated and 50 CTC-ITB-01 or 50 T47D (Cell line service, Eppelheim, Germany) cells were added to the PBMCs, as positive or negative control, respectively. The cell pellet was resuspended in 70 µl of MACS® buffer and 10 µl of FcR Blocking reagent (both Miltenyi Biotec, Bergisch Gladbach, Germany). The sample was then incubated with 20 µl of SUSD2-Micro Beads for 30 minutes at 4° C. in the dark. In the meantime, the LS column, containing ferromagnetic spheres, was placed in the VarioMACS™ Seperator (Miltenyi Biotec, Bergisch Gladbach, Germany), which is a permanent magnet that causes a high-gradient magnetic field within the column. The column was conditioned with 3 ml of MACS® buffer (Miltenyi Biotec, Bergisch Gladbach, Germany). The sample was diluted with 1 ml of MACS® buffer and then applied onto the column. The following washing step was performed for three times with 3 ml of MACS® buffer. For elution of labeled cells, the column was removed from the separator and cells were eluted with 3 ml of MACS® buffer and another 2 ml of MACS® buffer using the plunger to flush out all cells. One cytospin was generated from the combined flow-through and first washing fraction and another cytospin was obtained from the elution fraction. Patient samples were subjected to the same steps after PBMCs and tumor cells of patient blood samples were isolated with Ficoll Paque (GE Healthcare, Chalfont St Giles, United Kingdom). Volumes given above are for up to 10⁷ total cells. When working with more than 10⁷ cells, volumes were adjusted accordingly.

Recovery and detection rates were determined for the elution fraction and the combined flow-through and wash fraction, respectively (Table 2).

TABLE 2

Recovery and detection rates obtained with MACS® using SUSD2 MicroBeads and CTC-ITB-01 or T47D cells in spiking experiments (n=3)

| Cell line | Recovery rate in elution fraction | Detection rate in flow-through + wash fraction [%] |
|---|---|---|
| CTC-ITB-01 | 70.67 | 4.67 |
| T47D | 3.33 | 64.00 |

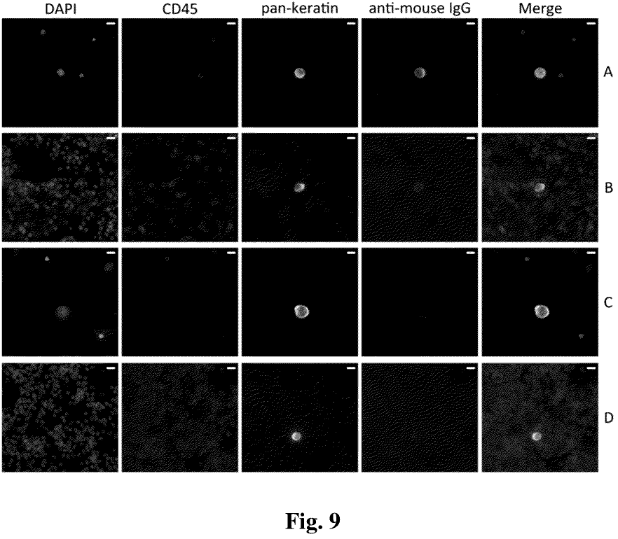

Cytospins, which were obtained after conduction of the MACS® spiking experiment using SUSD2 MicroBeads, were subjected to IF analysis (FIG. 9). As the SUSD2 MicroBeads are detectable with anti-mouse IgG antibody, the cytospins were stained with anti-mouse IgG to detect the SUSD2 MicroBeads bound to the cell surface. For the elution fraction of CTC-ITB-01 a circular staining was detected using an anti-mouse IgG antibody, indicating proper binding of the anti-SUSD2 MicroBeads to SUSD2 on the tumor cells. It was observed that very few CTC-ITB-01 cells also appear in the flow-through and wash fraction. However, these cells showed almost negative staining for mouse IgG, indicating no or imperfect binding of the MicroBeads to those cells. No signal was detected for T47D cells using anti-mouse IgG antibody, suggesting a high specificity of the SUSD2 MicroBeads. Nevertheless, very few T47D cells were found to appear in the elution fraction, detected with pan-keratin (Table 2).

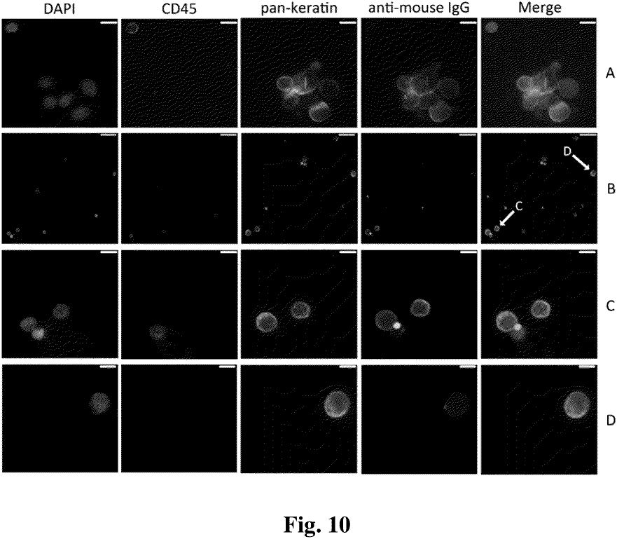

Blood samples of advanced stage breast cancer patients were subjected to the invention (SUSD2-MACS technology). FIG. 10 shows IF analysis of breast cancer patient CTCs obtained by MACS® using SUSD2 MicroBeads (elution fraction). Cytospin of the elution fraction was stained with antimouse lgG to detect CTCs bound by SUSD2 MicroBeads. Furthermore, pan-keratin was applied as tumor cell marker, CD45 as leukocyte marker and nuclei were visualized with DAPI. A: CTC cluster with SUSD2-positive CTCs. B: CTCs with positive and negative mouse- IgG staining. C: Zoom of CTCs with positive mouse IgG staining denoted by arrow C in image B. D: Zoom of CTCs with negative mouse IgG staining denoted by arrow D in image B. Images of the CTC cluster were taken at 400 × magnification and images in B at 200 × magnification. White scale bars represent 10 µm and blue scale bars correspond to 30 µm. In two patient samples enrichment of SUSD2-positive CTCs was achieved. Some CTCs were found to show a positive, circular staining for mouse-IgG, while others were negative (FIG. 10).

Accordingly, the above experimental data show that the inventors have successfully isolated and detected CTCs from the blood of a breast cancer patient with SUSD2 as biomarker using MACS. The same approach is also applicable to ENPP1. The procedure has been repeated to confirm detection of CTCs by approach 1 which shows that the isolated CTC were coupled to the magnetic nanoparticles confirming that these cells were specifically isolated by the magnetic nanoparticles and not due to unspecific carry-over from the blood sample. With approach 2, the presence of SUSD2 or ENPP1 can be confirmed by two different anti-SUSD2 or anti-ENPP1 specific antibodies. For CTC catching by MACS a first primary anti-SUSD2 antibody or anti-ENPP1 antibody 2 directed against the extracellular domain 11 has to be used and for SUSD2 or ENPP1 detection by immunofluorescence a second primary anti-SUSD2 or anti-ENPP1 specific antibody (4) directed against the cytoplasmic C-terminal tail of SUSD2 or ENPP1 has to be used.

Detection of keratin positive CTC confirms that the method of the invention is suitable for the specific isolation of CTC by SUSD2 or ENPP1. In addition, the method of the invention is suitable for the isolation of CTCs that are negative for keratin, thus CTC with mesenchymal attributes. It is reasonable that keratin negative cells that were positive for SUSD2 or ENPP1 are CTC with mesenchymal attributes. The invention provides a tool for the detection, isolation and molecular analysis of CTC with different characteristics, including mesenchymal attributes, which are considered as a probable candidate for the actual metastasis founding cells in breast cancer.

After confirmation of SUSD2 and ENPP1 in CTC of breast cancer patients, the inventors investigated DTC cell lines that were generated from the bone marrow of breast cancer patients. High levels of ENPP1 protein was detected in all analyzed DTC cell lines from breast, lung and prostate cancer patients. Notably, these cell lines showed no detectable signals for the epithelial marker proteins of the keratin family and EpCam. EpCam is most commonly used for the isolation of tumor cells from liquid samples by magnetic beads. Therefore, the analyzed DTC populations would remain undetectable by an anti-EpCam based MACS approach. Since DTC cell lines were positive for ENPP1, the invention could be used to enrich and isolate ENPPl-positive DTC from bone marrow samples. SUSD2 was detected in cell lines of different phenotype and could therefore be used for enrichment, isolation and detection of usually undetectable CTC/DTC but also of subpopulations that have an epithelial phenotype.

### CTC Biomarkers Candidates as Targets for Therapeutic Antibodies

Monoclonal antibodies are a rapidly growing class of drugs and the most common targets for therapeutic antibodies are cancers and autoimmune diseases. Antibodies can modulate signaling pathways, which promote tumor growth, survival and metastasis. Furthermore, antibodies bound to tumor cells can potentiate the host immune response against tumor cells by mediation of antibody-dependent cellular cytotoxicity, complement-dependent cytotoxicity or the induction of T-cell immunity. An ideal target of therapeutic monoclonal antibody in cancer should be a cell surface protein or a secreted protein mediating tumour-promoting effects. The CTC biomarker candidates identified during this work could be used as targets for therapeutic antibodies, since SUSD2 and ENPP1 are cell surface proteins. For cell surface proteins high and stable expression in tumor cells and a low or absent expression in normal tissues is desirable for targets of therapeutic antibodies, as this could decrease unwanted effects on normal cells and increase the concentration of unbound antibodies to affect the tumor cells. Furthermore, the pharmacological response is determined by the concentration of the antibody at the target. Despite an expression of the target by normal cells, a tumor-specific effect might be achieved if the bioavailability of the antibody is higher in the tumor tissue than in normal tissue. Antibodies are large proteins of approximately 150 kDa and might not reach the antigen in normal tissues due to organ-specific barriers but in the tumor tissue through leaky vessels. Therapeutic antibodies are commonly intravenously administered and might be primarily distributed in the blood, wherefore the target should not be expressed by normal blood cells.

SUSD2 is expressed by mesenchymal stem cells of the bone marrow, which might lead to unwanted side effects of therapeutic antibodies targeting SUSD2, as the bone marrow sinusoid capillaries are more permissive due to the fenestrated structure. Furthermore, SUSD2 is expressed in the essential organs like the lung or the kidney. Nevertheless, patients could be screened for SUSD2-positive CTCs with the CellSearch® system prior to application of such an antibody. In patients with high numbers of SUSD2-positive CTCs the antibody would likely bind to SUSD2 on CTCs in the blood stream and is no longer available to effect other cells. Advantageously, ENPP1 shows no or low expression in essential organs like the heart, lung or kidney. High expression of ENPP1 was found in the thyroid and parathyroid glands, which could be removed without life-threatening consequences. Therapeutic antibodies against ENPP1 might target tumor cells involved in therapy resistance (ABC transporter), wherefore the combination of an anti-ENPP1 antibody with existing therapies could improve patient outcome.

### REFERENCES

Bartkowiak, K.; Kwiatkowski, M.; Buck, F.; Gorges, T.M.; Nilse, L.; Assmann, V.; Andreas, A.; Muller, V.; Wikman, H.; Riethdorf, S., et al. Disseminated Tumor Cells Persist in the Bone Marrow of Breast Cancer Patients through Sustained Activation of the Unfolded Protein Response. Cancer Res 2015, 75, 5367-5377.

Koch, C., Kuske, A., Joosse, S. A., Yigit, G., et al., Characterization of circulating breast cancer cells with tumorigenic and metastatic capacity. EMBO Molecular Medicine 2020, 12, e11908.

Watson, A. P., Evans, R. L., Egland, K. A., Multiple functions of sushi domain containing 2 (SUSD2) in breast t umorigenesis. Mo/ Cancer Res 2013, 11, 74-85.

Hultgren, E. M., Patrick, M. E., Evans, R. L., Stoos, C. T., Egland, K. A., SUSD2 promotes tumor-associated macrophage recruitment by increasing levels of MCP-1 in breast cancer. PLoS One 2017, 12, e0177089.

Takahashi, R. -u., Miyazaki, H., Takeshita, F., Yamamoto, Y., et al., Loss of microRNA-27b contributes to breast cancer stem cell generation by activating ENPP1. Nature communications 2015, 6, 1-15.

Lau, W. M., Doucet, M., Stadel, R., Huang, D., et al., Enp pl : a potential facilitator of breast cancer bone metastasis. PLoS One 2013, 8, e66752.

Hu, M., Guo, W., Liao, Y., Xu, D., et al., Dysregulated ENPP1 increases the malignancy of human lung cancer by inducing epithelial-mesenchymal transition phenotypes and stem cell features. Am J Cancer Res 2019, 9, 134-144.

Lee, B.-J., Kang, D.-W., Park, H.-Y., Song, J.-S., et al., Isolation and localization of mesenchymal stem cells in human palatine tonsil by W5C5 (SUSD2) . Cellular Physiology and Biochemistry 2016, 38, 83-93.

Sivasubramaniyan, K., Harichandan, A., Schumann, S., Sobiesiak, M., et al., Prospective isolation of mesenchymal stem cells from human bone marrow using novel antibodies directed against Sushi domain containing 2. Stem cells and development 2013, 22, 1944-1954.

Masuda, H., Anwar, S.S., Buhring, H.J., Rao, J. R., Gargett, C. E., A novel marker of human endometrial mesenchymal stem-like cells. Cell Transplant 2012, 21, 2201-2214.

Kato K., Nishimasu H., Okudaira S., Mihara E., Ishitani R., Takagi J., Aoki J., Nureki O., 2012, Crystal structure of Enpp1, an extracellular glycoprotein involved in bone mineralization and insulin signaling, Proceedings of the National Academy of Sciences 109 (42), 16876-16881, DOI: 10.1073/pnas.1208017109

Onyedibe, K.I.; Wang, M.; Sintim, H.O. ENPP1, an Old Enzyme with New Functions, and Small Molecule Inhibitors-A STING in the Tale of ENPP1. Molecules 2019, 24, 4192, doi/10.3390/molecules24224192

Patrick, M.E.; Egland, K.A. SUSD2 Proteolytic Cleavage Requires the GDPH Sequence and Inter-Fragment Disulfide Bonds for Surface Presentation of Galectin-1 on Breast Cancer Cells. Int. J. Mol. Sci. 2019, 20, 3814. Doi:/10.3390/ijms20153814

Korsching, E.; Jeffrey, S.S.; Meinerz, W.; Decker, T.; Boecker, W., Buerger H. Basal carcinoma of the breast revisited: an old entity with new interpretations. J Clin Pathol. 2008 May;61(5):553-60. doi: 10.1136/jcp.2008.055475

Reference Numbers

| | |
|---|---|
| 1 | Transmembrane protein (SUSD2 or ENPP1) |
| 10 | signal peptide |
| 11 | extracellular domain |
| 110 | first (membrane bound) extracellular domain fragment |
| 111 | second extracellular domain fragment |
| 112 | extracellular domain cleavage site |
| 113 | disulfide bond |
| 12 | transmembrane domain |
| 13 | intracellular (cytoplasmic) domain |
| 14 | C-terminal |
| 15 | N-terminal |
| 2 | 1^{st} primary antibody (against SUSD2, ENPP1 extracellular domain; biotinylated); capturing antibody |
| 3 | 1^{st} secondary antibody (against 1^{st} primary antibody) |
| 4 | 2^{nd} primary antibody (against SUSD2, ENPP1 cytoplasmic domain) |
| 5 | 2^{nd} secondary antibody (against 2^{nd} primary antibody) |
| 50 | magnetic nanoparticle (with bound streptavidin) |
| 60 | cell membrane (of CTC/DTC) |
| 70 | MACS column |
| 71 | Magnet |
| 80 | CTC/DTC (SUSD2⁺, ENPP1⁺) |
| 81 | CTC/DTC (SUSD2⁻, ENPP1⁻) |
| 90 | collecting vessel |

I claim:

1. A method for isolation of circulating tumor cells (CTCs) and/or disseminated tumor cells (DTCs) from a blood, lymph or bone marrow sample of a subject, comprising:
a. obtaining from the subject a sample containing cells to be isolated,
b. exposing the cells to anti-SUSD2 or anti-ENPP1 antibodies conjugated to particles or a matrix having a separation functionality for a time sufficient for CTCs and/or DTCs to attach to the anti-SUSD2 or anti-ENPP1 antibody conjugated particles or matrix,
c. separating the particles or matrix from the sample using the separation functionality of the particles or matrix, thereby isolating CTCs and/or DTCs.

2. The method of claim 1, wherein the particles are magnetic nanoparticles, and wherein separating involves a magnetic field.

3. The method of claim 2, wherein the anti-SUSD2 or anti-ENPP1 antibodies conjugated to the magnetic nanoparticles are labelled with or contain at least one fluorophor.

4. The method of claim 1, wherein anti-SUSD2 or anti-ENPP1 antibodies are against full length SUSD2 or ENPP1.

5. The method of claim 1, wherein anti-SUSD2 or anti-ENPP1 antibodies are against truncated length SUSD2 or ENPP1.

6. The method of claim 1, wherein the matrix is an organic matrix having anti-SUSD2 or anti-ENPP1 antibodies coupled via reactive functional groups, and wherein separation involves sedimentation by gravity or centrifuge.

7. The method of claim 6, wherein the organic matrix is agarose.

8. The method as in claim 1, the CTCs or DTCs having a mesenchymal phenotype, wherein the CTCs or DTCs having a mesenchymal phenotype (mCTC, mDTC) have a predominant or exclusively mesenchymal phenotype, or a hybrid epithelial/mesenchymal phenotype (emCTC, emDTC).

9. The method of claim 1, wherein the anti-SUSD2 or anti-ENPP1 antibody conjugated particles are anti-SUSD2 conjugated particles.

10. The method of claim 1, wherein the anti-SUSD2 or anti-ENPP1 antibody conjugated particles are anti-ENPP1 conjugated particles.

11. A method for the isolation and detection of circulating tumor cells (CTCs) and/or disseminated tumor cells (DTCs) from a blood, lymph or bone marrow sample of a subject, comprising:
a. obtaining a blood, lymph or bone marrow sample from the subject,
b. exposing the cells to a first anti-SUSD2 or anti-ENPP1 antibody,
c. isolating any bound SUSD2 or ENPP1 positive cells from the sample,
d. exposing bound SUSD2 or ENPP1 positive cells to a second antibody for detection of bound SUSD2 or ENPP1 positive cells.

12. The method according to claim 11, wherein the second antibody is a labeled secondary antibody directed against the first anti-SUSD2 or anti-ENPP1 antibody, a second anti-SUSD2 or anti-ENPP1 antibody, or a labeled second anti-SUSD2 or anti-ENPP1 antibody, or a labeled secondary antibody directed against a second bound anti-SUSD2 or anti-ENPP1 antibody.

13. The method according to claim 11, further comprising isolation of T-cells from the blood of the subject, genetic modification of the T-cells to recognize SUSD2 or ENPP1 surface protein and re-injection of the genetic modified T-cells into the subject.

14. The method according to claim 11, wherein isolation is done immunomagnetically using anti-SUSD2 or anti-ENPP1 antibodies coupled directly or indirectly to magnetic nanoparticles.

15. The method according to claim 14, wherein cells labeled with anti-SUSD2 or anti-ENPP1 antibodies coupled to magnetic nanoparticles are applied to a ferromagnetic iron-column positioned in a magnetic field, wherein labeled cells are retained in the column and unlabeled or antigen-negative cells pass the column and are discarded, and wherein the ferromagnetic iron-column is removed from the magnetic field and the labeled cells are eluted and available for further analysis.

16. The method according to claim 11, wherein the circulating tumor cells (CTCs) and/or disseminated tumor cells (DTCs) have a mesenchymal phenotype, or a hybrid epithelial/mesenchymal phenotype.

17. The method of claim 11, wherein the anti-SUSD2 or anti-ENPP1 antibody is anti-SUSD2 antibody.

18. The method of claim 11, wherein the anti-SUSD2 or anti-ENPP1 antibody is anti-ENPP1 antibody.

19. A method for the isolation and detection of circulating tumor cells (CTCs) and/or disseminated tumor cells (DTCs) from a blood, lymph or bone marrow sample of a subject, comprising:
a. obtaining a sample containing cells to be tested,
b. exposing the cells to a first anti-SUSD2 or anti-ENPP1 antibody for capturing of the cells,
c. isolating any bound SUSD2 or ENPP1 positive cells from the sample,
d. exposing bound SUSD2 or ENPP1 positive cells to a second anti-SUSD2 or anti-ENPP1 antibody and/or an anti-keratin antibody for the immunofluorescent detection of bound SUSD2 or ENPP1 positive cells, wherein the anti-SUSD2 or anti-ENPP1 antibody is labelled with a different fluorophor from the anti-keratin antibody,
e. classifying SUSD2 or ENPP1/keratin double positive isolated cells as eCTC or emCTC and SUSD2 or ENPP1 positive/keratin negative cells as mCTC.


SEQUENCE LISTING (10 sequences)

SEQ ID NO: 1 - protein, 822 aa, Homo sapiens
MKPALLPWALLLLATALGPGPGPTADAQESCSMRCGALDGPCSCHPTCSGLGTCCLDFRDFCLEILPYSGSMMGGKDFVVRHFKMSSPTDASVICRFKDSIQTLGHVDSSGQVHCVSPLLYESGRIPFTVSLDNGHSFPRAGTWLAVHPNKVSMMEKSELVNETRWQYYGTANTSGNLSLTWHVKSLPTQTITIELWGYEETGMPYSQEWTAKWSYLYPLATHIPNSGSFTFTPKPAPPSYQRWRVGALRIIDSKNYAGQKDVQALWTNDHALAWHLSDDFREDPVAWARTQCQAWEELEDQLPNFLEELPDCPCTLTQARADSGRFFTDYGCDMEQGSVCTYHPGAVHCVRSVQASLRYGSGQQCCYTADGTQLLTADSSGGSTPDRGHDWGAPPFRTPPRVPSMSHWLYDVLSFYYCCLWAPDCPRYMQRRPSNDCRNYRPPRLASAFGDPHFVTFDGTNFTFNGRGEYVLLEAALTDLRVQARAQPGTMSNGTETRGTGLTAVAVQEGNSDVVEVRLANRTGGLEVLLNQEVLSFTEQSWMDLKGMFLSVAAGDRVSIMLASGAGLEVSVQGPFLSVSVLLPEKFLTHTHGLLGTLNNDPTDDFTLHSGRVLPPGTSPQELFLFGANWTVHNASSLLTYDSWFLVHNFLYQPKHDPTFEPLFPSETTLNPSLAQEAAKLCGDDHFCNFDVAATGSLSTGTATRVAHQLHQRRMQSLQPVVSCGWLAPPPNGQKEGNRYLAGSTIYFHCDNGYSLAGAETSTCQADGTWSSPTPKCQPGRSYAVLLGIIFGGLAVVAAVALVYVLLRRRKGNTHVWGAQP

SEQ ID NO: 2 - protein, 795 aa, Canis familiaris
MGSGQISSAAPPPGAQGSCSHRCGDRDGSCSCHPTCSGLGTCCSDFRDFCLEVSPYSGSMMGGKDFQVQHLTWFIPTDGVICRFKESIQTLGYVDSSGHVHCVSPLLYETGHIPFTLSMDNGRSFPRSGTWLAVHPSKVSETEKSQLENETLWQYYGTAGTTDNLTVTWEPSALPTRSVTIELWGYEETGKPYSGEWTAKWSYLYSLATSIPNSGFFTFTPKPAPPNFQRWEVGALRIISSRYYAGEKDVQALWSNEHALAWHLGEDFRADPKAWARAQCLAWEQLEDQLPNFLEELPDCPCTLAQARADSGRFHVSPPPPPPAPENQEGKGGEQCCYTEAGSLLLTADSTGGSTPDRGHDWGAPPFRTPPRVPGLSHWLYDVVSFYHCCLWAPECSRYMRRRPSSDCRSYRPSRLASAFGDPHFITFDGASFSFNGRGEYVLLEAELTNLRVQGRAQPRTTPEGPQDRGTGLTAVAVQEGNSDVVEVRLAGGAGVLQVLLNQEVLSFTEQSWMDLKGMFLSVAAGDSVSIMLSSGAGLEVSVQGPFLSVTVLLPEKFLTHTQGLLGTLNDDPTDDFVLRDGNVLPPKASSRELFRFGGLWTLGAVRNTSSLLTYDSWFLVNNFLYQPKHDYTFQPLFPEETTPNPSQAAEAAKLCGDNYFCIFDVMATGSLSVGNATRMAHQWHQHRAQSLQPVTSCGWLAPPPNGRKEGTRYLSGSTVYFHCDSGYSLVGAEVSTCQADGIWSRPTPMCQPARSYTVLLSIIFGGLAVVAVVALVYVLLRHRKGNMAIWGSQP

SEQ ID NO: 3 - protein, 823 aa, Cavia porcellus
MKLALLPWVLLLLATVPGPGPRPTADAQDSCSLRCGNQNGTCSCHPTCSGLGTCCPDFRDFCLEISPFSGSMMGGKDFVVRHFKWSTHTEGVICRFKESIQILGHVDSLGQVHCVSPLLYETGRIPFTISMDNGRSFLRAGTWLAVHPNKVSQSEKGQLVNETRWQYYGTPGTTTGNLSVTWDTSVLSTQTVNIELWGYEETGTPYSGNWIAKWSYLYSLATNAPNSGFFTFIPEPALASYQKWRVGALRITASKHYQGEKDVLAIWTNEHALAWHLGDDFRKDSVSWARAQCLDWEELEDNLPDFLKELPDCPCNLVQARADTGRFFTDYGCDIEQNSTCTYHPGAVHCVRSVQASPRFASGQQCCYRKDGSQLLTADSIGGSTPDRGHDWGAPPYRTPPRVPTLSHWLYDVMSFYYCCLWAPECSRYMQRRPSSDCRTYRPPRLASVFGDPHFVTFDGTKFTFNGRGEYVLLEGEADGKDLRVQARAEPTTLTSGTQARGTGLTAVVVQEDSSDVVEARLAHRPGVLEVLLNSEVLSFAEQSWMDLNGLFLSVAGEDKVSIMLSSGAGLEVSIQGPFLSVAVLLPEKFQNHTRGLLGTLNNNPADDFTLRSGMVLPSNASAQELFQFGAEWAVHNSSSLFTYDSWSLVYNFLYRPKHDSTFKPLFSDEIVLSPDQAEEVAKLCGDDHFCKFDVAVTGNLIVGNVTRAAHQMHQERLQSLQPVVSCGWLAAPSNGHKEDFKYLVGSTVRFHCESGYSLAGADTSTCQADGTWSAPTPECQLGRSYTVLLSIIFGGLAVVALVAIIYLLLRRRKNMNLWRSHP

SEQ ID NO: 4 - protein, 821 aa, Equus caballus
MKLLLLPWALLLLVTAPSPGRWPAADAQESCSHRCGEWNELCSCHPTCFGLDSCCKDFRDFCLEILPYSGSMMGGKDFVVQHLNWFSPTDGVICRFNESVQTRGHVDSLGHVHCVSPLLYETGRIPFTLSMDNGSSFPRSGTWLAVHPSKVSQTEKLQLENETRWQYYGTAGTKGNLTLTWNTAALPTQSVTIELWGYEETGKPYSEDWTAKWSYLYALATNVSNSGFFTFTPEPAPQNYQRWEVGALRIINSEYHSGEKDVHALWSNEHALAWHLGDDFRADPVAWARTQCLAWEELEDQLPNFLEELPDCPCTLAQARSDSGRFHTDYGCDIEHGSECTYHPGAVHCVRSVQASPQYASGQQCCYTAAGTQLLTADSMGGSTPDRGHDWGAPPFRAPPRVPGLSHWLYDVISFYHCCLWAPECSRYMRRRPSSDCRSYRPPRLASAFGDPHFVTFDGANFTFNGHGEYVLLEASLTNLTVQARAQPDTTPEGTQARGTGLTAVAVQEGDSDVVEVRLAGGAGVLQVLLNQEVLSFAEQSWMDLKGMFLSVAAEDRVSIMLSSGAGLEVSIQGPFLSVTVLLPEKFLTYTQGLLGTLNDNFTDDFTLRSGQVLPPDASSQELFQFGADWAVENASSLLTYDSQLLVNNFLYGPKHDPTFRPLFPDEITPNSSQTTEVAKLCGDNHFCSFDVAATGSLSVGNATRMAYQLHQRRVQSLQSVVSCGWLAPPLNGHKDGMRYLVGSTIHFHCDTGYSLAGAEASTCQANGTWSTPTPTCQPRRSHTVLLSIIFGGLAVVALMAVVFVVLRRRKGNMAIWGSQP

SEQ ID NO: 5 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 6 - protein, 916 aa, Canis familiaris
MERGGQAGLGSREGPAGNGPDPGCARAAAAPGDGQAAAALLAPMDLGEEPLEKAARARPAKDPNTYKVLSLVLSVCVLTTILGCIFGLKPSCAKEVKSCKGRCFERTFGNCRCDVACVDLGNCCLDYQETCIEPERIWTCSKFRCGEKRLSRSLCSCSDDCRDKGDCCVNYSSVCLGEKSWVEETCESIDEPQCPAGFEMPPTLLFSLDGFRAEYLHTWGGLLPVISKLKNCGTYTKNMRPVYPTKTFPNHYSIVTGLYPESHGIIDNKIYDPKMNAFFALKSKEKFNPEWYKGEPIWLTTKYQGLKSGTFFWPGSDVEIKGILPDIYKMYNGSIPFEERILAVLKWLQLPKDERPHFYTLYLEEPDSSGHSYGPVSSEVIRALQRVDNMVGMLMDGLKGLNLHRCLNLILISDHGMEQGSCKKYVYLNKYLGDVKDIKIVYGPAARLRPSDVPNKYFSFNYEDLAKNLSCREPNQHFKPYLKHFLPKRMHFAKNDRIEPLTFYLDPQWQLALNPSERKHCGSGFHGSDNLFSNMQALFIGYGPGFKHNIEVDSFENIEVYNLMCDLLNLTPAPNNGTHGSLNHLLKNPVYTPKNPKEVHPMVQCPFTRAPRHNLDCSCDPSVLPVTDFETQLNLTVAEEKVIKRGTLPYGRPRVIQKNTTVCLLYQHQFVSGYSHDILMPLWTSYTVDRNDSFSTEDFSNCLYQDLRIPLSPIHKCSFYKNNAELSYGFLYPPQLNKGSNQLYSEALLSTNIVPMYQSFQVIWHYFHGTLLQRYVEERNGVNVVSGPVFDSDYDGRYDSSETLRQNSRLIHNQEILIPTHFFIVLTNCKDTFQTPSQCDDLDSLAFILPHRADNSESCVHGKHESLWVEELLRLHRARITDVEHLTGLSFYQERRESISDILKLKTQLPAFDQED

SEQ ID NO: 7 - protein, 900 aa, Cavia porcellus
MEQRDGGDAGRAGPAGDPQAAAALLTPMELGEEPLEKAARARTAKDPNTYKVLSLVLSVCVLTTILGCIFGLKPSCSREVKSCKGRCFERTFGNCRCDSACVDFGNCCLDYQETCLEPAHIWTCNKFRCGEKRVPHNLCSCSDDCKDHDDCCVNYSSMCQDEKSWLEAPCERINTPQCPAGFDAPPTLLFSLDGFRAEYLHTWGGLLPVISKLKNCGTYTKNMRPVYPTKTFPNHYTIVTGLYPESHGIIDNRMFDPQMNASFSFKTKQKFNPQWYQGEPIWLTAKYQHLISGTFFWPGSDVEINGNFPDFYEVYNSSIPFEKRILGVLDWLQLPQGERPHFYTLYLEEPDSAGHFYGPVSGEVIKALQRVDSMVGMLMEGLRELKLDKCLNLLLVSDHGMEQDSCKKYVYLNKYLGDVTNIKVTYGPAARLRLSDVLNEYSTFDYEGLARNLSCREPKQHFKPYLKQFLPKRFHFANNDRIEPLTFYLDPQWQLALDPTERKHCGGGFHGSDNIFSNMQALFIGYGPGFKHGVEVDTFENIELYNLMCDLLKLTPAPNNGTHGSLNHLLKHPVYTPKHPKEAQSLGKCPITGTPRDGLGCSCDPAISPVEDFQIQFNLTRTEEKNIDRGALPYGRPRLVQRKGSVCLLYHHQFVSGYSLDILMPLWASYTVDKNDRFSVGDFSNCVFQDLRIPLSPLHKCSFYKNNAKLSYGFLAPPQLSKYASQIYSEALLTTNIVPMYPSFQVIWRYLHDTLLPRLAKEKNGINVVSGPVFDSDFDGHSDTAETLKQNRRFIRNQEVLIPTHFFMVLTSCKNTLGTPLYCTELDTMAFILPHRPDNSESCTHEKSESSWIEELLRMHRARVIDVEAITGLSFYQERKQPVSDILKLKTHLPIFSEED

SEQ ID NO: 8 - protein, 1043 aa, Equus caballus
MACADHTLRFQSRPRRQATQGDSCKCADGAQGASRGGWGEGQDGPTQRTRNNRHAGSPKSDRKPNNPGRRVAFPSLGGRGAGAGPGVRRAGPGRAGRGGRLGLLKARRGGGPSRVSMERDGCAGGGSHGGGCEGGRGSREDPAGNGRDPSRGHASPAPGDPQAAASLLAPMDVGEEPLEKAARAGAAKDPNTYKVLSLVLSVCVLTVILGCIFGLKPSCAKEVKSCKGRCFERTFGNCRCDSACADLGNCCLDYQETCIEPERIWTCSKFRCGEKRLSRSLCSCSDDCRDKDDCCINYSSACLGEKSWVQETCENINEPQCPAGFEMPPTLLFSLDGFRAEYLHTWGGLLPVINKLKTCGTYAKNMRPVYPTKTFPNHYSIVTGLYPESHGIIDNKMYDPKMNASFALKSKEKFNPKWYKGEPIWLTAKYQGLKAGTFFWPGSDVKINGLFPDIYKIYNGSVPFEERILAVLKWLQLPKDERPHFYTLYLEEPDSSGHSHGPVSSEVIRALQRVDNMVGMLMDGLKGLNLHRCLNLILISDHGMEQASCKKYAHLDKYLGDVKDIKLVYGPAARLRPSDVPDKYYSFNYEGLARNLSCRELNQHFKPYLKHFLPKRLHFAKNDRIEPLTFYLDPQWQLALNPPAEKHCQGGFHGSDNVFSNMQALFIGYGPGFQHGAEVDSFENIEVYNLMCDLLNLIPASNNGTHGSLNHLLKNPIYTPTHPKEVSSLVQCPFTRTPRNNLGCSCNPSILPIVDLQTQLNLTMAEENIIKRGTLPYGRPRVIQKNSTVCLLYQHQFVSGYSHDILMPLWTSYTVDRNDIFSTEDFSNCLYQDLRVSLSPVHECSFYKNNAKLSYGFLSPPQLNKGSSKINSEALLTTNIVPMYQSFQVIWHYFHGTLLQRYAQERNGVNVVSGPVFDSDFDGRYDSSETLKQNSRRVRNQEVLIPTHFFIVLTSCKDPDQAPSQCENLDASAFILPHRTDNSESCVHGKQESSWVEELLRLHRARITDVEQITGLSFYQERKEPVSDILKLKTHLPIFNQED

SEQ ID NO: 9 - protein, 25 aa, Artificial Sequence
  SUSD2 peptide
CGALDGPCSCHPTCSGLGTCCLDFR

SEQ ID NO: 10 - protein, 16 aa, Artificial Sequence
  ENPP1 peptide
AEYLHTWGGLLPVISK